SEQUENCE LISTING (48 sequences)

SEQ ID NO: 1 - DNA, 20 bp, Artificial Sequence
  Description of Artificial Sequence Synthetic primer
tccagtctgtgcgagaggta

SEQ ID NO: 2 - DNA, 20 bp, Artificial Sequence
  Description of Artificial Sequence Synthetic primer
acggatgaagatggcgatag

SEQ ID NO: 3 - DNA, 21 bp, Artificial Sequence
  Description of Artificial Sequence Synthetic primer
cgagaggtagcagagcagaga

SEQ ID NO: 4 - DNA, 20 bp, Artificial Sequence
  Description of Artificial Sequence Synthetic primer
aagcactccgtgaactcctg

SEQ ID NO: 5 - DNA, 20 bp, Artificial Sequence
  Description of Artificial Sequence Synthetic primer
ttacgccccaaagtctgttt

SEQ ID NO: 6 - DNA, 20 bp, Artificial Sequence
  Description of Artificial Sequence Synthetic primer
cccatcagccatctgtatca

SEQ ID NO: 7 - DNA, 20 bp, Artificial Sequence
  Description of Artificial Sequence Synthetic primer
gagtctcagctggtggcaat

SEQ ID NO: 8 - DNA, 22 bp, Artificial Sequence
  Description of Artificial Sequence Synthetic primer
tgcacatctccataaacacctc

SEQ ID NO: 9 - DNA, 20 bp, Artificial Sequence
  Description of Artificial Sequence Synthetic primer
tccagactcagcagttgtgg

SEQ ID NO: 10 - DNA, 24 bp, Artificial Sequence
  Description of Artificial Sequence Synthetic primer
tctcatcaggaataactcttcagc

SEQ ID NO: 11 - DNA, 19 bp, Artificial Sequence
  Description of Artificial Sequence Synthetic primer
ggctgaaccagggaaaaga

SEQ ID NO: 12 - DNA, 20 bp, Artificial Sequence
  Description of Artificial Sequence Synthetic primer
ggagcttgacagcttcagga

SEQ ID NO: 13 - DNA, 18 bp, Artificial Sequence
  Description of Artificial Sequence Synthetic primer
aagcagaacgggattccc

SEQ ID NO: 14 - DNA, 21 bp, Artificial Sequence
  Description of Artificial Sequence Synthetic primer
tggaatcagaaagcactacgc

SEQ ID NO: 15 - DNA, 20 bp, Artificial Sequence
  Description of Artificial Sequence Synthetic primer
gagtcttccttggagcgaga

SEQ ID NO: 16 - DNA, 20 bp, Artificial Sequence
  Description of Artificial Sequence Synthetic primer
caacctcagtggacaaggtg

SEQ ID NO: 17 - DNA, 20 bp, Artificial Sequence
  Description of Artificial Sequence Synthetic primer
cctcatattctgcgggatga

SEQ ID NO: 18 - DNA, 21 bp, Artificial Sequence
  Description of Artificial Sequence Synthetic primer
ggtcgcagtgtatgtttgatg

SEQ ID NO: 19 - DNA, 19 bp, Artificial Sequence
  Description of Artificial Sequence Synthetic primer
ggcgtttgttggggttact

SEQ ID NO: 20 - DNA, 18 bp, Artificial Sequence
  Description of Artificial Sequence Synthetic primer
ctgagtccgcagcaggtg

SEQ ID NO: 21 - DNA, 20 bp, Artificial Sequence
  Description of Artificial Sequence Synthetic primer
ggcaacagtgtcagagtcca

SEQ ID NO: 22 - DNA, 20 bp, Artificial Sequence
  Description of Artificial Sequence Synthetic primer
ctgaggcgtatttgggaaag

SEQ ID NO: 23 - DNA, 20 bp, Artificial Sequence
  Description of Artificial Sequence Synthetic primer
cagcatctttggttgcttgt

SEQ ID NO: 24 - DNA, 20 bp, Artificial Sequence
  Description of Artificial Sequence Synthetic primer
ccgacaatatgctcaacgtg

SEQ ID NO: 25 - DNA, 20 bp, Artificial Sequence
  Description of Artificial Sequence Synthetic primer
ctcgaaagaagccaccagag

SEQ ID NO: 26 - DNA, 20 bp, Artificial Sequence
  Description of Artificial Sequence Synthetic primer
gcagaaggggcaaagtagag

SEQ ID NO: 27 - DNA, 20 bp, Artificial Sequence
  Description of Artificial Sequence Synthetic primer
gcagccatcttattccgttc

SEQ ID NO: 28 - DNA, 20 bp, Artificial Sequence
  Description of Artificial Sequence Synthetic primer
gaaaagtagcccccaacctc

SEQ ID NO: 29 - DNA, 20 bp, Artificial Sequence
  Description of Artificial Sequence Synthetic primer
gggacacagctttcacccta

SEQ ID NO: 30 - DNA, 21 bp, Artificial Sequence
  Description of Artificial Sequence Synthetic primer
ggggcaatctggtatatgttg

SEQ ID NO: 31 - DNA, 21 bp, Artificial Sequence
  Description of Artificial Sequence Synthetic primer
ttcactgcagaggaagcatct

SEQ ID NO: 32 - DNA, 20 bp, Artificial Sequence
  Description of Artificial Sequence Synthetic primer
ggtctggttccttggtttca

SEQ ID NO: 33 - DNA, 19 bp, Artificial Sequence
  Description of Artificial Sequence Synthetic primer
ggtcccactggaagaggtc

SEQ ID NO: 34 - DNA, 19 bp, Artificial Sequence
  Description of Artificial Sequence Synthetic primer
ccagcgctcttacttggtg

SEQ ID NO: 35 - DNA, 21 bp, Artificial Sequence
  Description of Artificial Sequence Synthetic primer
acgcaggacagaatgatgaag

SEQ ID NO: 36 - DNA, 20 bp, Artificial Sequence
  Description of Artificial Sequence Synthetic primer
tgcgacttcaacagcaactc

SEQ ID NO: 37 - DNA, 20 bp, Artificial Sequence
  Description of Artificial Sequence Synthetic primer
cttgctcagtgtccttgctg

SEQ ID NO: 38 - DNA, 19 bp, Artificial Sequence
  Description of Artificial Sequence Synthetic primer
gcttgcgagctgcatttgg

SEQ ID NO: 39 - DNA, 22 bp, Artificial Sequence
  Description of Artificial Sequence Synthetic primer
catctccagttgtcctcttcca

SEQ ID NO: 40 - DNA, 20 bp, Artificial Sequence
  Description of Artificial Sequence Synthetic primer
tctgtgcgagaggtagcaga

SEQ ID NO: 41 - DNA, 21 bp, Artificial Sequence
  Description of Artificial Sequence Synthetic primer
tggccgggtctgctgagtccg

SEQ ID NO: 42 - DNA, 21 bp, Artificial Sequence
  Description of Artificial Sequence Synthetic primer
gtccatgggaagatgttctgg

SEQ ID NO: 43 - DNA, 20 bp, Artificial Sequence
  Description of Artificial Sequence Synthetic primer
ccaggtgcatccaatttttc

SEQ ID NO: 44 - DNA, 20 bp, Artificial Sequence
  Description of Artificial Sequence Synthetic primer
gcctggctgttcattgtttc

SEQ ID NO: 45 - DNA, 20 bp, Artificial Sequence
  Description of Artificial Sequence Synthetic primer
cagcagctgccaaacttcta

SEQ ID NO: 46 - DNA, 20 bp, Artificial Sequence
  Description of Artificial Sequence Synthetic primer
cattgcatctgttggtgagg

SEQ ID NO: 47 - DNA, 20 bp, Artificial Sequence
  Description of Artificial Sequence Synthetic primer
cagcaatgcaagctgctagt

SEQ ID NO: 48 - DNA, 20 bp, Artificial Sequence
  Description of Artificial Sequence Synthetic primer
tgggatgacaaccaaaatcc

(19) United States
(12) Patent Application Publication
(10) Pub. No.: US 2018/0010188 A1
(43) Pub. Date: Jan. 11, 2018

(54) METHOD FOR DETECTING NONSENSE-MEDIATED RNA DECAY

(71) Applicant: The Regents of the University of California, Oakland, CA (US)

(72) Inventor: Sika Zheng, Riverside, CA (US)

(73) Assignee: The Regents of the University of California, Oakland, CA (US)

(21) Appl. No.: 15/644,226

(22) Filed: Jul. 7, 2017

Related U.S. Application Data
(60) Provisional application No. 62/359,377, filed on Jul. 7, 2016.

(51) Int. Cl. C12Q 1/68; G01N 33/50

(52) U.S. Cl. CPC C12Q 1/6883; G01N 33/5023; C12Q 2600/158

(57) ABSTRACT

Certain embodiments of the invention provide a method of detecting and/or quantitating nonsense mediated RNA decay (NMD) activity in a cell, comprising 1) detecting an altered level of RNA expression of a NMD-sensitive isoform in a cell, as compared to a control cell; and 2) detecting an unaltered level of RNA expression of a corresponding NMD-insensitive isoform in the cell, as compared to a control cell, wherein the isoforms are derived from an endogenously expressed, alternatively spliced gene.

### RELATED APPLICATION

This application claims the benefit of priority of U.S. Provisional Application Ser. No. 62/359,377 filed on Jul. 7,2016, which application is incorporated by reference herein.

### GOVERNMENT FUNDING

This invention was made with Government support under Grant No. MH096807, awarded by the National Institutes of Health. The Government has certain rights in the invention.

### BACKGROUND OF THE INVENTION

Nonsense-mediated RNA decay (NMD) is a post-transcriptional quality control mechanism that ensures the fidelity of cellular gene expression by selectively degrading aberrant transcripts containing a premature stop codon (PTC) (Popp M W-L, Maquat L E. 2013. Annu Rev Genet 47: 139-165; Rebbapragada I, Lykke-Andersen J. 2009. Curr Opin Cell Biol 21: 394-402; Chang et al., 2007. Annu Rev Biochem 76: 51-74; Lykke-Andersen S, Jensen T H. 2015. Nat Rev Mol Cell Biol 16: 665-677). Because of cellular NMD activity, nonsense mutations often lead to the loss of protein products, which accounts for the molecular pathogenesis of over 20% of monogenic diseases (Mort et al., 2008. Hum Mutat 29: 1037-1047; Bidou, et al., 2012. Trends Mol Med 18: 679-688; Holbrook et al., 2004. Nat Genet 36: 801-808; Kurosaki T, Maquat L E. 2016. J Cell Sci 129: 461-467). Recent studies also support a role of NMD as an essential gene regulation mechanism that quantitatively determines the abundance of physiologic transcripts with NMD features (Peccarelli M, Kebaara B W. 2014. Eukaryot Cell 13: 1126-1135; Chan et al., 2007. EMBO J 26: 1820-1830; Isken O, Maquat L E. 2008. Nat Rev Genet 9: 699-712; Rehwinkel et al., 2005. RNA 11: 1530-1544; Nott et al., 2003. RNA 9: 607-617). The depletion of core NMD factors leads to the upregulation of many transcripts (Mendell et al., 2004. Nat Genet 36: 1073-1078; Wittmann et al., 2006. Mol Cell Biol 26: 1272-1287; Schmidt et al., 2015. Nucleic Acids Res 43: 309-323; Weischenfeldt et al., 2012. Genome Biol 13: R35). Most strikingly, snoRNA host genes and some alternatively spliced NMD-sensitive isoforms are consistently repressed by NMD in various cell types and organs (Lykke-Andersen et al., 2014. Genes Dev 28: 2498-2517; Thoren et al., 2010. PLoS One 5: e11650; Weischenfeldt et al., 2008. Genes Dev 22: 1381-1396; Lykke-Andersen S, Jensen T H. 2015. Nat Rev Mol Cell Biol 16: 665-677). The coupling of alternative splicing and NMD (AS-NMD), or regulated unproductive splicing and translation (RUST), is widely used by splicing regulators and some RNA binding proteins (RBP) to maintain their own homeostatic expression (Lareau et al., The Coupling of Alternative Splicing and Nonsense-Mediated mRNA Decay. compbio.berkeley.edu/people/brenner/pubs/lareau-2007-landes-nmd.pdf; Ni et al., 2007. Genes Dev 21: 708-718; Saltzman et al., 2008. Mol Cell Biol 28: 4320-4330; Boutz et al., 2007. Genes Dev 21: 1636-1652; Spellman et al., 2007. Mol Cell 27: 420-434). AS-NMD is also harnessed to control the expression of non-RBP genes during development (Zheng et al., 2012. Nat Neurosci 15: 381-8, 51; Zheng S. 2016. Int Dev Neurosci. dx.doi.org/10.1016/j.ijdevneu.2016.03.003). NMD regulation of endogenous genes is integrated into a variety of physiological settings including the fine-tuning of the unfolded protein response (UPR) (Sakaki et al., 2012. Proc Natl Acad Sci USA 109: 8079-8084; Oren et al. 2014. EMBO Mol Med 6: 685-701; Karam et al., 2015. EMBO Rep 16: 599-609; Lou et al., 2014. Cell Rep 6: 748-764; Maquat L E, Gong C. 2009. Biochem Soc Trans 37: 1287-1292). Despite extensive studies on the molecular mechanisms of target recognition and decay, less is known about the control of NMD activity. Several studies have shown that NMD can be inhibited as an adaptive response to hypoxia and calcium signaling (Nickless et al. 2014. Nat Med 20: 961-966; Gardner L B. 2008. Mol Cell Biol 28: 3729-3741) or in the tumor microenvironment (Wang et al., 2011. Mol Cell Biol 31: 3670-3680).

Given the essential role of NMD in disease pathogenesis and gene expression regulation, methods that precisely detect changes in NMD activity are of great interest. Traditional methods often rely on a pair of plasmid reporters, with one containing a PTC and the other lacking a PTC. This reporter pair is engineered to normalize the impact of transcription and other regulatory mechanisms affecting transcript abundance in order to isolate NMD regulation. Because the two contrasting reporters are often separately delivered into the cultured cells and their transcripts assayed individually, cell-to-cell variation adds to the noise in the attempt to differentiate the two reporters. To mitigate these effects, a third control plasmid is usually co-transfected along with the two reporters. Alternatively, some reporter pairs rely on varying protein readouts as the proxies of the NMD and non-NMD transcript levels. This approach potentially introduces false positives reflecting differences in translation initiation and/or protein stability rather than differences in NMD activity itself.

Exogenous reporter assays are invaluable in characterizing NMD regulatory mechanisms, but their application is often restricted to transfectable cells. Issues concerning delivery route and efficiency as well as the need for comparison between multiple plasmids present substantial obstacles for a broader application of plasmid reporters in primary cells and animals. Furthermore, despite the careful measures to enhance the precision of these reporter assays, their robustness is unavoidably affected by many variables inherent to a general reporter gene approach. The degree of overexpression, the transfection method, the variation of transfection efficiency, the quantity and quality of reporter DNA, the choice of the control plasmid and cell density all affect the reporter readout and must be painstakingly controlled to preserve the signal-to-noise ratio and consistency. Cell lines stably expressing a reporter gene can mitigate variation induced by transient transfection, but they also introduce new variables, such as integration loci and copy numbers, which unpredictably affect reporter readout (Zheng et al., 2013. Genome Res 23: 998-1007). These limitations are prohibitive for adopting PTC reporter assays for unbiased screens.

Accordingly, there is a need for new methods for detecting NMD.

### SUMMARY OF THE INVENTION

Thus, described herein is a sensitive and quantitative perturbation-free method for detecting genuine NMD activity using one or more (e.g., a panel of) endogenous NMD targets.

Certain embodiments of the invention provide a method of detecting and/or quantitating nonsense mediated RNA decay (NMD) activity in a cell, comprising measuring the RNA expression level of a NMD-sensitive isoform and a corresponding NMD-insensitive control isoform in the cell, wherein the isoforms are derived from an endogenously expressed, alternatively spliced gene, so as to detect and/or quantitate NMD activity.

Certain embodiments of the invention provide a method of detecting and/or quantitating nonsense mediated RNA decay (NMD) activity in a cell, comprising 1) detecting an altered level of RNA expression of a NMD-sensitive isoform in a cell, as compared to a control cell; and 2) detecting an unaltered level of RNA expression of a corresponding NMD-insensitive isoform in the cell, as compared to a control cell, wherein the isoforms are derived from an endogenously expressed, alternatively spliced gene.

Certain embodiments of the invention provide a method for measuring the presence of a biomarker in a cell, the improvement comprising:

1) measuring whether the RNA expression level of a NMD-sensitive isoform in a cell is altered as compared that in a control cell; and

2) measuring whether the RNA expression level of a corresponding NMD-insensitive isoform in the cell is unaltered as compared to that in a control cell;

wherein the isoforms are derived from an endogenously expressed, alternatively spliced gene;

for use in detecting and/or quantitating nonsense mediated RNA decay (NMD) in the cell.

As described herein, NMD activity may be inferred when 1) an altered level of RNA expression of a NMD-sensitive isoform in a cell, as compared to a control cell is detected; and 2) an unaltered level of RNA expression of a corresponding NMD-insensitive isoform in the cell, as compared to a control cell is detected.

Certain embodiments of the invention provide a method for screening a compound for nonsense mediated RNA decay (NMD) modulating activity, comprising

1) measuring the RNA expression level of a NMD-sensitive isoform and a corresponding NMD-insensitive isoform in a first population of cells, wherein the isoforms are derived from an endogenously expressed, alternatively spliced gene;

2) contacting a second population of cells with the compound; and

3) subsequently measuring the RNA expression level of the NMD-sensitive isoform and the corresponding NMD-insensitive isoform in the second population of cells;

wherein the compound has NMD modulating activity if i) the RNA expression level of the NMD-sensitive isoform in the second population of cells is altered as compared to the first population of cells; and ii) the RNA expression level of the corresponding NMD-insensitive isoform in the second population of cells is unaltered as compared to the first population of cells.

Certain embodiments of the invention provide a method of detecting and/or quantitating nonsense mediated RNA decay (NMD) activity in a cell, comprising 1) detecting an altered level of RNA expression of at least one NMD-sensitive isoform in a cell, as compared to a control cell; and 2) detecting an unaltered level of RNA expression of at least one corresponding NMD-insensitive isoform in the cell, as compared to a control cell, wherein the isoforms are derived from an endogenously expressed, alternatively spliced gene selected from Ptbp2, Hnrnpl, Srsf11, Tra2b and/or Psd-95; and wherein the RNA expression levels are detected using real-time quantitative polymerase chain reaction (RT-qPCR).

Certain embodiments of the invention provide a kit for detecting and/or quantitating nonsense mediated RNA decay (NMD) activity in a cell, comprising 1) two or more primer pairs for detecting/measuring the RNA expression level of a NMD-sensitive isoform and a corresponding NMD-insensitive isoform in the cell, wherein the isoforms are derived from an endogenously expressed, alternatively spliced gene; and 2) and instructions for using the primers to detect/measure the RNA expression of the isoforms.

Certain embodiments of the invention provide a method of inhibiting nonsense mediated RNA decay (NMD) in a cell, comprising contacting the cell with an effective amount of thapsigargin, or a salt thereof.

### BRIEF DESCRIPTION OF THE FIGURES

FIGS. 1A-G. Unlike traditional alternative splicing assays, the method described herein can on its own distinguish NMD activity from alternative splicing regulation. (A) Schematics of the RT-qPCR method specifically detecting alternative splicing isoforms. The NMD isoform can be either the inclusion or exclusion isoform, while the other isoform is designated as the non-NMD isoform. The inclusion isoform is detected by primer F1 and isoform-specific primer R1. The exclusion isoform is detected by F2 and isoform-specific junction primer R2. Primers F3 and R3 are commonly used in alternative splicing assays to detect both inclusion and exclusion isoforms. (B) Conventional splicing assay derived expression ratios of the Psd-95 NMD isoform relative to the non-NMD isoform in N2a cells depleted of UPF1 or overexpressing PTBP1. The ratios by themselves do not distinguish NMD regulation (caused by UPF1 depletion) from alternative splicing regulation (caused by PTBP1 overexpression). Representative digital gel images are shown in the lower panel. A one-way ANOVA test was used to determine significant ratio changes between different samples, followed by a Tukey's multiple comparison test. N=3. (C) RT-qPCR analysis of the same samples as in (B) using different primers specific to the Psd-95 non-NMD isoform (left) and Psd-95 NMD (NPsd-95) isoform (right). A two-way ANOVA test followed by Dunnett's multiple comparison tests was used to determine significant expression changes between samples. N=3. (D) SiUpf1 knockdown efficiency validated by RT-qPCR. (E) Alternative splicing assay of the Psd-95 NMD and non-NMD isoforms in Upf2 conditional knockout cortices (left panel) and Ptbp2 mutant cortices (right panel). This assay cannot possibly conclude whether ratio changes between the two isoforms are due to NMD regulation (e.g., Upf2 knockout) or alternative splicing regulation (e.g., Ptbp2 knockout). Statistics were calculated using a two-tailed unpaired Student's t test for the Upf2 knockout samples (N=2) and a one-way ANOVA test followed by Tukey's multiple comparison test for the Ptbp2 mutant samples (Ptbp2^{+/+} and Ptbp2^{+/-}, n=2; Ptbp2^{-/-}, n=4). (F-G) RT-qPCR analysis of the same samples as in (E) using different primers specific to the Psd-95 non-NMD isoform (left) and Psd-95 NMD (NPsd-95) isoform (right). This assay unambiguously concludes that ratio changes between the two isoforms are due to NMD regulation in the case of Upf2 knockout and alternative splicing regulation in the case of Ptbp2 knockout. A two-way ANOVA test followed by Dunnett's multiple comparison tests was used to determine significant expression changes between samples. *, P=(0.01, 0.05); **, P=(0.001, 0.01); ***, P=(0.0001, 0.001); ****, P<0.0001. Error bars represent mean±SEM.

FIGS. 2A-E. Thapsigargin specifically stabilizes endogenous NMD targets. (A-E) Temporal expression profiles of NMD isoforms (squares) and non-NMD isoforms (circles) of Srsf11, Ptbp2, Tra2b, Hnrnpl and Psd-95 upon 0.2 μM thapsigargin treatment. Expression levels are normalized to DMSO-treated samples. A two-way ANOVA test was used to determine significant difference between isoform levels. ###, P=(0.0001, 0.001); ####, P<0.0001. Dunnett's multiple comparison tests were used to determine significant expression changes of the NMD or non-NMD isoforms independently over time, e.g., DMSO vs. 1 hr, DMSO vs. 2 hr, etc. *, P=(0.01, 0.05); **, P=(0.001, 0.01); ***, P=(0.0001, 0.001); ****, P<0.0001. N=3. Error bars represent mean±SEM.

FIGS. 3A-D. Dose-dependent correlation between ER stress, polysome disassembly and NMD inhibition upon thapsigargin treatment. (A) Time course analysis of Xbp1 splicing (top panel) upon application of 0.2 μM thapsigargin in N2a cells. N=3. (B) Thapsigargin dose-dependent Xbp1 splicing in N2a cells at 5 hours after treatment. N=3. (C) Thapsigargin dose-dependent expression of the NMD (squares) and non-NMD (circles) isoforms of Srsf11, Ptbp2, Tra2b, Hnrnpl and Psd-95 in N2a cells at 5 hours after treatment. A two-way ANOVA test was used to determine significant difference between the two isoforms. ##, P=(0.001, 0.01); ####, P<0.0001. Dunnett's multiple comparison tests were used to determine significant expression changes of the NMD or non-NMD isoforms independently in comparison to DMSO treatment, e.g., DMSO vs. 0.01 μM, DMSO vs. 0.02 μM, etc. *, P=(0.01, 0.05); **, P=(0.001, 0.01); ***, P=(0.0001, 0.001); ****, P<0.0001. N=3. Error bars represent mean±SEM. (D) Polysome fractionation graphs of N2a cells at 5 hours after treatment with DMSO, 0.01 μM, 0.02 μM, 0.05 μM, 0.1 μM and 0.2 μM thapsigargin (TG). 40 s, 60 s, 80 s, disome (black arrow) and polysome are labeled accordingly. In each graph, a dashed line is drawn from the disome peak to the peak of the 8-ribosome fraction.

FIGS. 4A-K. PERK is necessary for thapsigargin-induced NMD inhibition. Expression levels of Perk (A); NMD and non-NMD isoforms of Psd-95 (B), Ptbp2 (C) and Tra2b (D); and Xbp1s (E) and Bip (F) after siPerk knockdown and thapsigargin treatment. Within (B)-(D) the NMD isoform is shown on the right and the non-NMD isoform is shown on the left for each grouping. Control siRNA and two different siRNAs targeting Perk were transfected into N2a cells for 48 hours before thapsigargin application. Note that the thapsigargin effect on the NMD isoforms was completely blocked by siPerk transfection (B-D). Expression levels of the NMD and non-NMD isoforms of Psd-95 (G), Ptbp2 (H) and Tra2b (I) as well as Xbp1s (J) and Bip (K) in N2a cells treated with DMSO, thapsigargin or thapsigargin plus PERK inhibitor GSK2606414 at various concentrations. Within (G)-(I) the NMD isoform is shown on the right and the non-NMD isoform is shown on the left for each grouping. A concentration of 0.1 μM GSK2606414 or above was sufficient to revert the effect of thapsigargin on the NMD isoforms (G-I). A one-way ANOVA test was used for A, E, F, J and K. A two-way ANOVA test followed by Dunnett's multiple comparison tests was used for B, C, D, G, H and I. ***, P=(0.0001, 0.001); ****, P<0.0001; ns: not significant. N=3. Error bars represent mean±SEM.

FIGS. 5A-E. Thapsigargin enhances TDP-43-repressed cryptic splicing isoforms through PERK. Expression of the normal and cryptic isoforms of A230046K03Rik (A), Mib1 (B) and Ups15 (C) as well as the expression levels of Tdp-43 (D) and Perk (E) in N2a cells transfected with control siRNA, siTdp43 and/or siPerk and subsequently treated with DMSO or 0.2 μM thapsigargin. Within each grouping for (A)-(E), thapsigargin is shown on the right and DMSO is shown on the left. Representative digital gels are shown in the lower panels. Arrows point to the cryptic and normal isoforms with the indicated amplicon sizes. The ratio of the cryptic isoform relative to the normal isoform was quantified for each gene under each experimental condition (upper panels). In siTdp43 cells, thapsigargin further increased the ratios. In siTdp43 and siPerk double-knockdown cells, thapsigargin had no effect on the ratios compared to DMSO. ns, P≧0.05; *, P=(0.01, 0.05); **, P=(0.001, 0.01); ***, P=(0.0001, 0.001); ****, P<0.0001 (two-way ANOVA followed by Dunnett's multiple comparison tests). All error bars represent mean±SEM.

FIGS. 6A-B. (A) In scenarios where NMD isoform levels increase, assaying the non-NMD isoforms distinguishes NMD regulation from transcriptional and alternative splicing regulation. (B) In scenarios where NMD isoform levels decrease, assaying the non-NMD isoforms distinguishes NMD regulation from transcriptional and alternative splicing regulation.

FIGS. 7A-D. Schematics of exon-skipping junction primers annealing to and amplifying (A) exclusion isoforms and (B-D) inclusion isoforms.

FIGS. 8A-B. Ouabain did not induce significant changes in NMD targets. RT-qPCR assay of (A) immediate early genes, c-fos and Pip92, as well as (B) the NMD (right) and non-NMD isoforms (left) of Psd-95, Ptbp2 and Hnrnpl in N2a cells treated with ouabain. Isoform specific primers were used for the expression assay. Ouabain clearly induced c-fos and Pip92 but did not unequivocally affect NMD activity.

FIGS. 9A-C. Deprivation of L-glutamine inhibits NMD. Expression levels of the NMD (right) and non-NMD (left) isoforms of Psd-95 (A), Ptbp2 (B) and Tra2b (C) in N2a cells cultured in L-glutamine-free media for 12 and 15 hours. The NMD isoforms but not the non-NMD isoforms were significantly upregulated by L-glutamine deprivation. A two-way ANOVA followed by Dunnett's multiple comparison tests was used to determine significant changes in gene expression. *, P=(0.01, 0.05); ***, P=(0.0001, 0.001); ****, P<0.0001. N=3. Error bars represent mean±SEM.

FIG. 10. RT-qPCR primers.

FIG. 11. RT-PCR primers for splicing assay.

### DETAILED DESCRIPTION

Studies on NMD have previously relied on exogenous reporter pairs. However, reporter assays are cumbersome, prone to high experimental variations, limited by delivery efficiency and do not necessarily mirror endogenous regulation. As described herein, a new strategy to reliably and conveniently monitor NMD activity has been designed, which overcomes several previously problematic aspects of using endogenous targets to report NMD activity. The method describes herein measures the abundance of one or more (e.g., a panel of) endogenous alternatively spliced NMD isoforms and their non-NMD counterparts. Changes in NMD activity are directly inferred and distinguished from transcriptional and other posttranscriptional regulatory mechanisms. This method provides a real time sensitive measurement of cellular NMD activity with a broad dynamic range.

This method was subsequently used to test pharmacological inhibitors for their effects on the expression of these targets. A potent inhibitor, thapsigargin, which inhibited NMD at a concentration as low as 20 nM, was identified. This highly sensitive method also allowed the determination of the molecular mechanism of thapsigargin's inhibitory action.

Methods of Detecting and/or Quantitating Nonsense Mediated RNA Decay

Alternative splicing is a regulated process during gene expression that results in a single gene coding for multiple isoforms. In this process, particular exons of a gene may be included within or excluded from the final, processed messenger RNA (mRNA) produced from that gene. Accordingly, multiple mRNA isoforms may be generated for a particular gene, wherein each isoform comprises a different set of exons. Particular isoforms may be subject to NMD and are termed “NMD-sensitive isoforms”, whereas other isoforms are not subject to NMD and are termed “NMD-insensitive isoforms”. Because the two isoforms are identical with the exception of the alternative exonic material, they should be subject to the same regulation other than NMD. Altering NMD activity should change the abundance of the NMD isoform but not the abundance of the non-NMD isoform. Accordingly, as the abundance of the NMD-insensitive isoform does not change when NMD activity is altered, this isoform may be used as a control for comparison to the NMD-sensitive isoform. In contrast, transcriptional regulation elevates or lowers the levels of both isoforms in the same direction and changes in alternative splicing alter the amount of the two isoforms in the opposite directions. Therefore, measuring and comparing the levels of the NMD-sensitive and NMD-insensitive isoforms from the same gene can be used to effectively infer NMD activity.

Genes that are alternatively spliced, wherein at least one isoform is targeted by NMD and at least one isoform is not targeted by NMD, are known in the art (i.e., an alternative splicing-induced NMD (AS-NMD) gene target). Isoforms from such genes may be used in the invention described herein. For example, polypyrimidine tract binding protein 2 (Ptbp2, nPTB or brPTB; NM_019550) is an RNA binding protein that regulates alternative splicing. Skipping exon 10 shifts the reading frame of Ptbp2 and generates an isoform sensitive to NMD (Boutz et al., 2007. Genes Dev 21: 1636-1652; Spellman et al., 2007. Mol Cell 27: 420-434). Including exon 10 produces a NMD-insensitive isoform. Other such AS-NMD gene targets include, but are not limited to, heterogeneous nuclear ribonucleoprotein L (Hnrnpl; NM 177301), serine/arginine-rich splicing factor 11 (Srsf11, Sfrs11; NM_001093752), transformer 2 beta (Tra2b; NM_009186) and postsynaptic density protein 95 (Psd-95, Dlg4; NM 007864). The NMD transcript isoforms of these genes are as follows: Hnrnpl including exon 6, Srsf11 including exon 2, Tra2b including exon 2 and Psd-95 excluding exon 18 (Saltzman et al., 2008. Mol Cell Biol 28: 4320-4330; Spellman et al., 2007. Mol Cell 27: 420-434; Zheng et al., 2013. Genome Res 23: 998-1007; Stoilov et al., 2004. Hum Mol Genet 13: 509-524). Accordingly, endogenously expressed, alternatively spliced gene(s) include, but are not limited to, Ptbp2, Hnrnpl, Srsf11, Tra2b and Psd-95; however, the methods of the invention may examine any other gene(s) that is alternatively spliced, wherein at least one isoform is targeted by NMD and at least one isoform is not targeted by NMD.

Accordingly, certain embodiments of the invention provide a method of detecting and/or quantitating nonsense mediated RNA decay (NMD) activity in a cell, comprising measuring the RNA expression level of a NMD-sensitive isoform and a corresponding NMD-insensitive isoform in the cell, wherein the isoforms are derived from an endogenously expressed, alternatively spliced gene, so as to detect and/or quantitate NMD activity.

Certain embodiments of the invention also provide a method of detecting and/or quantitating nonsense mediated RNA decay (NMD) activity in a cell, comprising 1) detecting an altered level of RNA expression of a NMD-sensitive isoform in a cell, as compared to a control cell; and 2) detecting an unaltered level of RNA expression of a corresponding NMD-insensitive isoform in the cell, as compared to a control cell, wherein the isoforms are derived from an endogenously expressed, alternatively spliced gene, so as to detect and/or quantitate NMD activity.

Certain embodiments of the invention provide a method of detecting and/or quantitating nonsense mediated RNA decay (NMD) activity in a cell, comprising measuring the RNA expression level of at least one NMD-sensitive isoform and at least one corresponding NMD-insensitive isoform in the cell, wherein the isoforms are derived from an endogenously expressed, alternatively spliced gene, and wherein an altered level of RNA expression of the NMD-sensitive isoform and an unaltered level of RNA expression of the corresponding NMD-insensitive isoform, as compared to a control cell, is indicative of NMD activity.

Certain embodiments of the invention provide a method for measuring the presence of a biomarker in a cell, the improvement comprising:

1) measuring whether the RNA expression level of a NMD-sensitive isoform in a cell is altered as compared to a control cell; and

2) measuring whether the RNA expression level of a corresponding NMD-insensitive isoform in the cell unaltered as compared to a control cell;

wherein the isoforms are derived from an endogenously expressed, alternatively spliced gene;

for use in detecting and/or quantitating nonsense mediated RNA decay (NMD) in the cell.

The phrase “altered level of RNA expression” refers to the level of RNA expression in cells or organisms that differs from that of control cells or organisms (e.g., a normal cell/organism or a cell/organism not exposed to a test condition, such as a compound or a change in time (e.g., a developmentally different stage)). In certain embodiments, the level of RNA expression is increased, e.g., increased by about 10%, 15%, 20%, 25%, 30%, 35%, 40%, 45%, 50%, 55%, 60%, 65%, 70%, 75%, 80%, 85%, 90%, 95%, 100% or more, as compared to that in a control cell/organism. In certain embodiments, the level of RNA expression is decreased, e.g., decreased by about 10%, 15%, 20%, 25%, 30%, 35%, 40%, 45%, 50%, 55%, 60%, 65%, 70%, 75%, 80%, 85%, 90%, 95%, 100% or more, as compared to that in a control cell/organism.

The phrase “unaltered level of RNA expression” refers to the level of RNA expression in cells or organisms that is about the same as that from control cells or organisms (e.g., a normal cell/organism or a cell/organism not exposed to a test condition, such as a compound). As used herein the phrase “about the same” means less than ±10%.

Certain embodiments of the invention also provide a method for screening a compound for nonsense mediated RNA decay (NMD) modulating activity, comprising 1) measuring the RNA expression level of at least one NMD-sensitive isoform and at least one corresponding NMD-insensitive isoform in a first population of cells, wherein the isoforms are derived from an endogenously expressed, alternatively spliced gene; 2) contacting a second population of cells with the compound; and 3) subsequently measuring the RNA expression level of the at least one NMD-sensitive isoform and the at least one corresponding NMD-insensitive isoform in the second population of cells, wherein the compound has NMD modulating activity if i) the RNA expression level of the at least one NMD-sensitive isoform in the second population of cells is altered as compared to the first population of cells; and ii) the RNA expression level of the at least one corresponding NMD-insensitive isoform in the second population of cells unaltered as compared to the first population of cells.

Typically, the first and second populations of cells are the same cell type and have been maintained under similar conditions. This will enable any effect from the compound to be more clearly ascertained.

The phrase “NMD modulating activity” refers to the ability of a compound to increase or decrease NMD activity in a cell. In certain embodiments, a compound may increase NMD activity in a cell by, e.g., about 5%, 10%, 15%, 20%, 25%, 30%, 35%, 40%, 45%, 50%, 55%, 60%, 65%, 70%, 75%, 80%, 85%, 90%, 95%, 100% or more, as compared to a control cell/organism not exposed to the compound. In such an embodiment, the RNA expression level of the NMD-sensitive isoform would be decreased and the RNA expression level of the NMD-insensitive isoform would be unaltered. In certain embodiments, a compound may decrease NMD activity in a cell by, e.g., about 5%, 10%, 15%, 20%, 25%, 30%, 35%, 40%, 45%, 50%, 55%, 60%, 65%, 70%, 75%, 80%, 85%, 90%, 95%, 100% or more, as compared to a control cell/organism not exposed to the compound. In such an embodiment, the RNA expression level of the NMD-sensitive isoform would be increased and the RNA expression level of the NMD-insensitive isoform would be unaltered.

In certain embodiments, the RNA expression level of the NMD-sensitive isoform and the corresponding NMD-insensitive isoform in the second population of cells is measured about 5, 10, 15, 20, 25, 30, 35, 40, 45, 50, 55, 60, 90 or 120 min after contact with the compound. In certain embodiments, the RNA expression level of the NMD-sensitive isoform and the corresponding NMD-insensitive isoform in the second population of cells is measured about 2, 3, 4, 5, 6, 7, 8, 9, 10, 11, 12, 18, 24 or more hours after contact with the compound.

In certain embodiments, a method of the invention comprises detecting/measuring the RNA expression levels of NMD-sensitive and corresponding insensitive isoforms derived from two or more endogenously expressed, alternatively spliced genes e.g., 2, 3, 4, 5, 6 or more AS-NMD targets, such as a panel of AS-NMD targets) (i.e., a NMD sensitive and a corresponding NMD-insensitive isoform are assayed for each gene). In certain embodiments, a panel of endogenously expressed, alternatively spliced genes is evaluated.

In certain embodiments, the endogenously expressed, alternatively spliced gene(s) comprises a stop codon >50 nts upstream of an exon-exon junction.

In certain embodiments, the endogenously expressed, alternatively spliced gene(s) is/are Ptbp2, Hnrnpl, Srsf11, Tra2b and/or Psd-95, or any combination thereof.

In certain embodiments, the NMD-sensitive and NMD-insensitive isoforms differ by a cassette exon (e.g., a short cassette exon). In certain embodiments, the NMD-sensitive and NMD-insensitive isoforms do not have alternative 5′UTRs, alternative 3′UTRs and/or intron retention differences.

In certain embodiments, the methods of the invention further comprise converting the RNA isoforms into cDNA prior to detecting/measuring the expression levels.

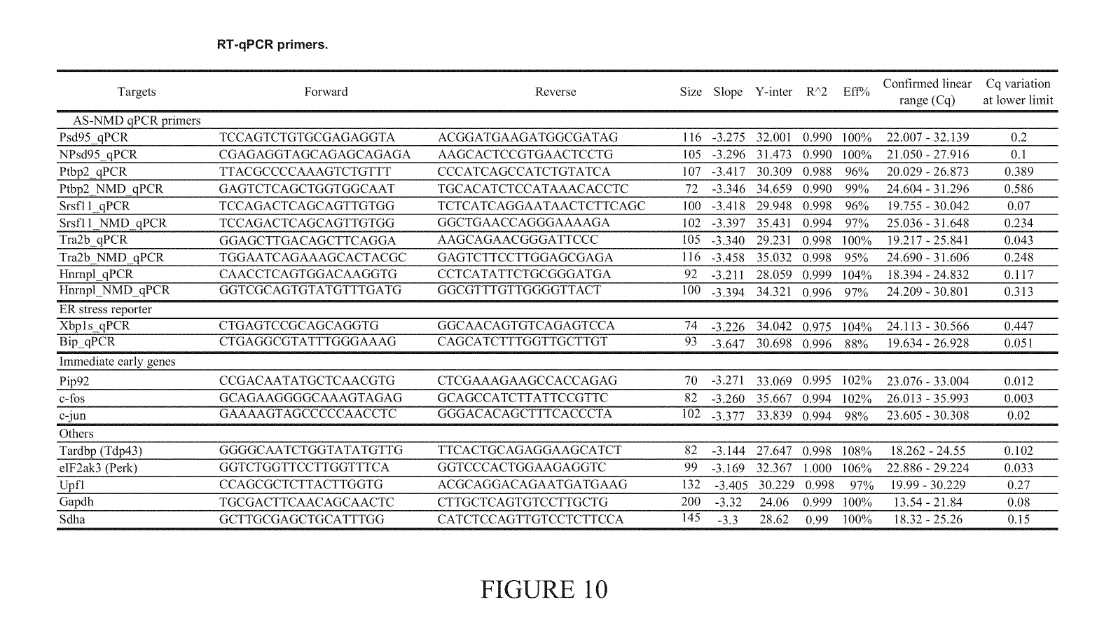

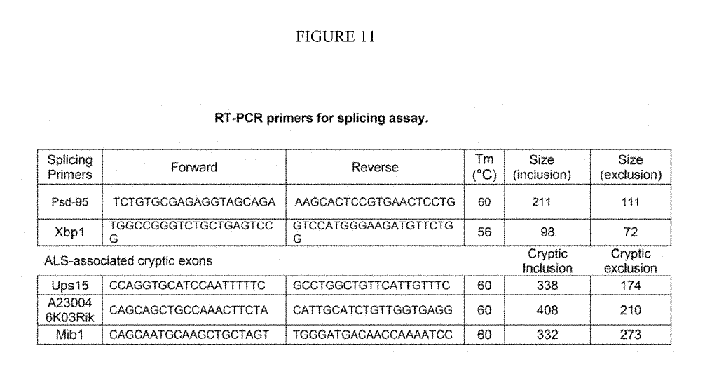

Methods for detecting/measuring RNA expression levels are known in the art. In certain embodiments, the RNA expression levels are detected/measured using real-time polymerase chain reaction (qPCR). In certain embodiments, the RNA expression levels are detected/measured using real-time quantitative polymerase chain reaction (RT-qPCR). In certain embodiments, the RNA expression levels are detected/measured using digital PCR. In certain embodiments, the RNA expression levels are detected/measured using high-throughput sequencing (e.g., next generation sequencing, third generation sequencing, fourth generation sequencing, etc.). In certain embodiments, an assay as described herein is used to detect/measure the RNA expression levels of the various isoforms. In certain embodiments, one or more primers as described herein (e.g., FIG. 10) are used in the assay for detecting/measuring the RNA expression levels. In certain embodiments, one or more primers having at least 75%, 80%, 85%, 86%, 87%, 88%, 89%, 90%, 91%, 92%, 93%, 94%, 95%, 96%, 97%, 98%, 99% or 100% sequence identity to a primer as described herein (e.g., FIG. 10) is used in the assay for detecting/measuring the RNA expression levels.

In certain embodiments, the methods further comprise normalizing the RNA expression levels to the expression of one or more housekeeping genes (e.g., Gapdh or Sdha).

In certain embodiments, the RNA expression levels of the NMD-sensitive and NMD-insensitive isoforms are separately detected/measured. For example, in certain embodiments, NMD-sensitive and NMD-insensitive isoforms are amplified separately. As discussed in the Example, this is distinct from an alternative splicing assay, which simultaneously amplifies both isoforms in one reaction and then resolves the two isoforms by electrophoresis. Such a semi-quantitative assay cannot definitively distinguish NMD regulation from alternative splicing regulation.

Accordingly, in certain embodiments, a method described herein is capable of distinguishing NMD activity from both alternative splicing regulation and transcriptional regulation.

Certain embodiments of the invention provide a method of detecting and/or quantitating nonsense mediated RNA decay (NMD) activity in a cell, comprising 1) detecting an altered level of RNA expression of at least one NMD-sensitive isoform in a cell, as compared to a control cell; and 2) detecting an unaltered level of RNA expression of at least one corresponding NMD-insensitive isoform in the cell, as compared to a control cell, wherein the isoforms are derived from an endogenously expressed, alternatively spliced gene selected from Ptbp2, Hnrnpl, Srsf11, Tra2b and/or Psd-95; and wherein the RNA expression levels are detected using real-time quantitative polymerase chain reaction (RT-qPCR).

In certain embodiments, the cell(s) are mammalian cells (e.g., such as cells from a human, mouse, rat, dog, cat, hamster, guinea pig, rabbit, livestock, and the like.). In certain embodiments, the cell(s) are mouse cells. In certain embodiments, the cell(s) are human cells.

In certain embodiments, the cell(s) is a primary cell(s) or immortalized cell(s). In certain embodiments, the cell(s) is comprised within a tissue or an organ. In certain embodiments, the cell(s) is in a sample (e.g., a biological sample) obtained from a subject (e.g., a mammal, such as a human). In certain embodiments, the sample is a postmortem sample. In certain embodiments, the sample is from a living subject. As used herein, a biological sample includes both biological fluids and tissues.

In certain embodiments, a method of the invention further comprises obtaining one or more cells (e.g., a plurality of cells) from a mammal. In certain embodiments, a method of the invention further comprises obtaining a biological sample comprising one or more cells (e.g., a plurality of cells) from a mammal.

In certain embodiments, a method of the invention further comprises contacting the cell with a compound having NMD modulating activity (e.g., thapsigargin).

As described herein, the methods of the invention may also be used as a reporter assay for detecting ER stress and/or translation inhibition. For example, certain embodiments of the invention provide a method of detecting and/or quantitating ER stress and/or translation inhibition in a cell, comprising measuring the RNA expression level of a NMD-sensitive isoform and a corresponding NMD-insensitive isoform in the cell, wherein the isoforms are derived from an endogenously expressed, alternatively spliced gene, so as to detect and/or quantitate ER stress and/or translation inhibition.

Thus, methods of the invention may be used to test the impact of a certain condition on NMD activity. Accordingly, certain embodiments of the invention provide a method for screening a test condition for nonsense mediated RNA decay (NMD) modulating activity, comprising 1) measuring the RNA expression level of at least one NMD-sensitive isoform and at least one corresponding NMD-insensitive isoform in a first population of cells, wherein the isoforms are derived from an endogenously expressed, alternatively spliced gene; 2) exposing a second population of cells to the test condition; and 3) subsequently measuring the RNA expression level of the at least one NMD-sensitive isoform and the at least one corresponding NMD-insensitive isoform in the second population of cells, wherein the test condition has NMD modulating activity if i) the RNA expression level of the at least one NMD-sensitive isoform in the second population of cells is altered as compared to the first population of cells; and ii) the RNA expression level of the at least one corresponding NMD-insensitive isoform in the second population of cells unaltered as compared to the first population of cells. Test conditions include, but are not limited to, e.g., environmental conditions, such as temperature, nutrients, and oxygen/carbon dioxide levels, cell density, developmental stage, time, genetic status, etc.

### Kits

The present invention further provides kits for practicing the present methods. Accordingly, certain embodiments of the invention provide a kit for detecting and/or quantitating nonsense mediated RNA decay (NMD) activity in a cell, comprising 1) one or more reagents for detecting/measuring the RNA expression level of a NMD-sensitive isoform and a corresponding NMD-insensitive isoform in the cell, wherein the isoforms are derived from an endogenously expressed, alternatively spliced gene; and 2) and instructions for use. Such kits may optionally contain one or more of: a positive and/or negative control, RNase-free water, and one or more buffers. In certain embodiments, a kit may further include RNase-free laboratory plasticware.

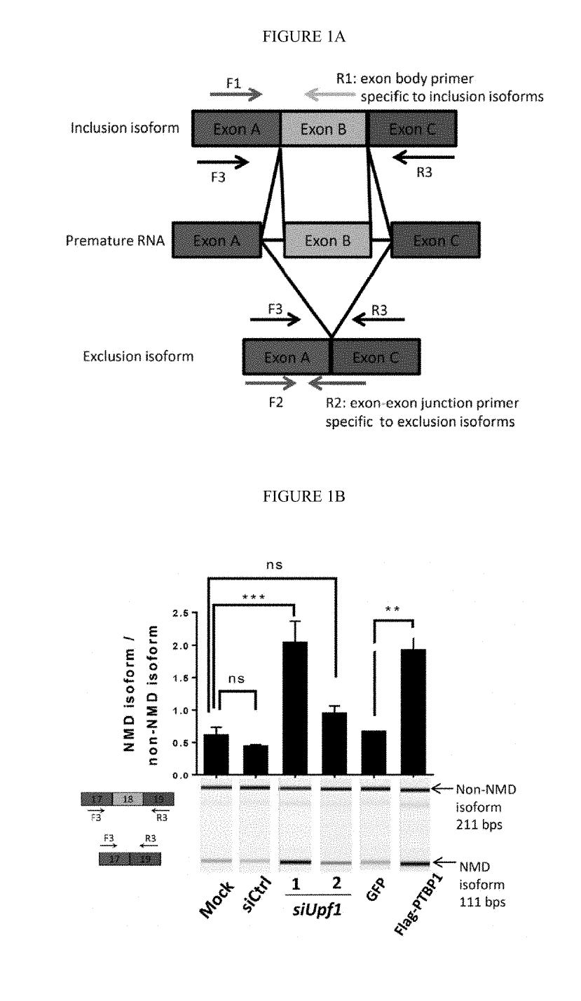

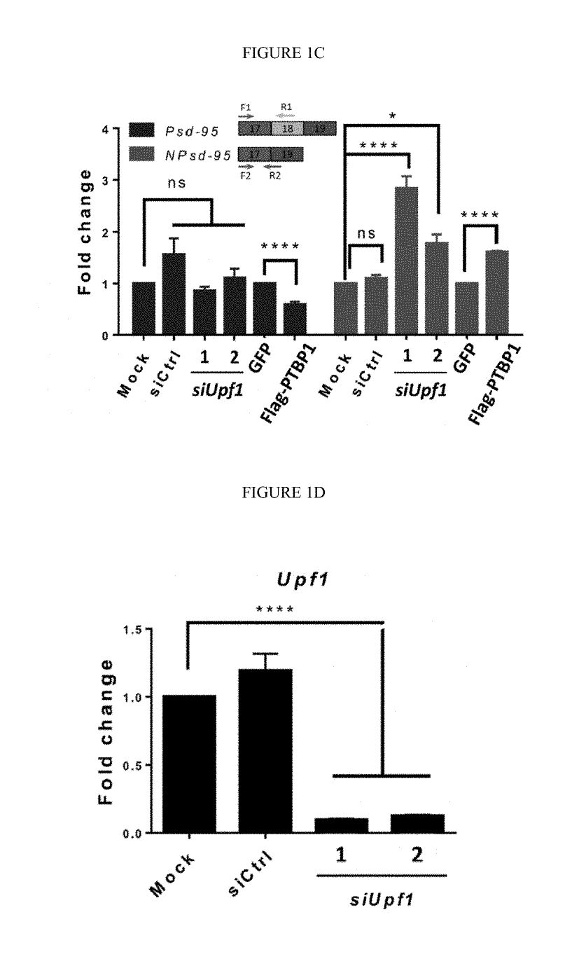

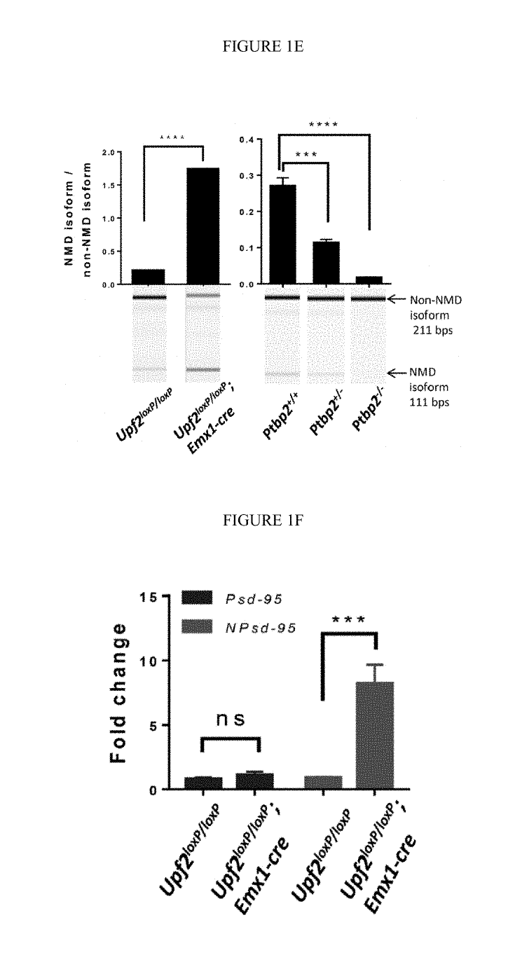

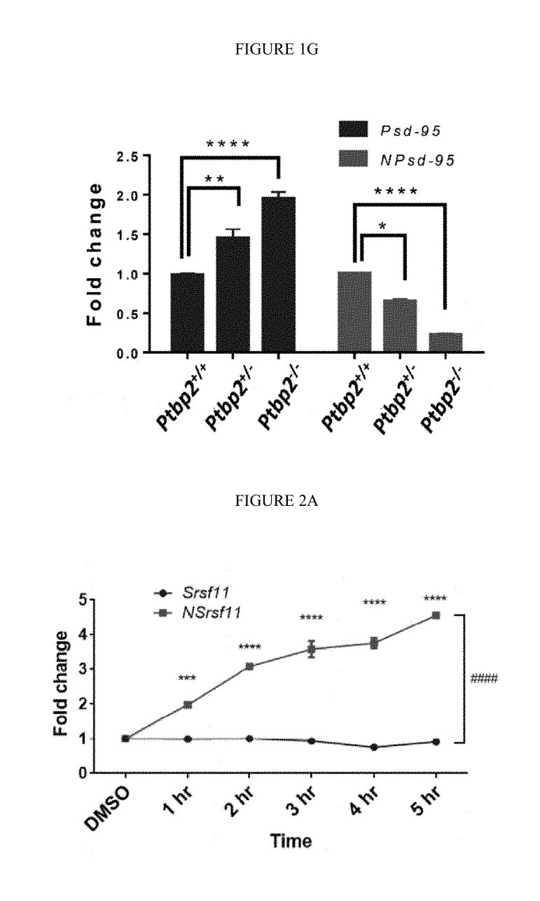

In certain embodiments, the one or more reagents are one or more primers. In certain embodiments, the kit may contain a number of primers that is any integer between 1 and 100, such as 1, 2, 3, 4, 5, 6, 7, 8, 9, 10, . . . 100. As used herein, the term “nucleic acid primer” encompasses both DNA and RNA primers. In certain embodiments, the primer(s) is labeled (e.g., fluorescently labeled). In certain embodiments, the primers may be used in a RT-qPCR assay. For example, in certain embodiments, the kit comprises primers for specifically amplifying an NMD-sensitive isoform and primers for specifically amplifying a corresponding NMD-insensitive isoform for each endogenously expressed, alternatively spliced gene to be evaluated. In certain embodiments, the kit comprises primer pairs for specifically amplifying the NMD-sensitive and insensitive isoforms of Ptbp2, Hnrnpl, Srsf11, Tra2b and/or Psd-95, or any combination thereof. In certain embodiments, the kit comprises one or more primers as described in FIG. 10. In certain embodiments, the kit comprises one or more primers having at least 75%, 80%, 85%, 86%, 87%, 88%, 89%, 90%, 91%, 92%, 93%, 94%, 95%, 96%, 97%, 98%, 99% or 100% sequence identity to a primer as described herein (e.g., FIG. 10). In certain embodiments, the primers are designed as described herein (e.g., as described in FIG. 1A). For example, an inclusion isoform may be amplified using a primer pair, wherein one of the primers is specific to the alternative exon, and the skipping isoform may be amplified using a primer pair, wherein one of the primers is specific to the exon-exon junction of the two flanking constitutive exons. In certain embodiments, at least one primer is not considered a product of nature. For example, in certain embodiments, at least one primer comprises a sequence that is not found in nature. In certain embodiments, at least one primer comprises a non-natural modification. For example, in certain embodiments, at least one primer is conjugated to a label, such as a fluorescent label or a radioactive label. In certain embodiments, at least one primer comprises a non-natural nucleotide or comprises a non-natural backbone modification.

### Methods of Inhibiting NMD

Certain embodiments of the invention provide a method of inhibiting nonsense mediated RNA decay (NMD) in a cell, comprising contacting the cell with an effective amount of thapsigargin, or a salt thereof.

Certain embodiments of the invention provide a method of inhibiting nonsense mediated RNA decay (NMD) in a cell, comprising contacting the cell with an effective amount of thapsigargin, or a salt thereof, wherein the cell had been determined to have NMD activity using a method described herein.

In certain embodiments, the cell is a primary cell, an immortalized cell or in a tissue organ.

In certain embodiments, the cell is a mammalian cell (e.g., such as a cell from a human, mouse, rat, dog, cat, hamster, guinea pig, rabbit, livestock, and the like.). In certain embodiments, the cell is a mouse cell. In certain embodiments, the cell is a human cell.

In certain embodiments, the mammal has a disease or disorder associated with NMD. Diseases and/or disorders associated with nonsense mediated RNA decay (NMD) are known in the art, and include, but are not limited to Duchenne muscular dystrophy, Cystic fibrosis, ataxia-telangiectasia-like disorder, Hurler's syndrome, Frasier syndrome, Ulrich's disease and MRXS14. In certain embodiments, the disease or disorder associated with nonsense mediated RNA decay (NMD) may be a monogenic disease/disorder caused by a nonsense mutation.

In certain embodiments, the cell is contacted with thapsigargin in vitro.

In certain embodiments, NMD activity in the cell is reduced by, e.g., about 5%, 10%, 15%, 20%, 25%, 30%, 35%, 40%, 45%, 50%, 55%, 60%, 65%, 70%, 75%, 80%, 85%, 90%, 95%, 100% or more, as compared to a control cell not exposed thapsigargin, or a salt thereof.

### Certain Definitions

A “gene,” for the purposes of the present disclosure, includes a DNA region encoding a gene product, as well as all DNA regions which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses mRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

A “coding sequence,” or a sequence that “encodes” a selected polypeptide, is a nucleic acid molecule that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and especially synthetic DNA sequences. A transcription termination sequence may be located 3′ to the coding sequence.

The term “endogenous gene” refers to a native gene in its natural location in the genome of an organism.

The term “RNA expression” refers to the transcription of a gene (e.g., an endogenous gene) and the accumulation of messenger RNA (mRNA) in cells.

“Oligonucleotide probe” can refer to a nucleic acid segment, such as a primer, that is useful to amplify a sequence in the gene of interest that is complementary to, and hybridizes specifically to, a particular sequence in the gene of interest. Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254, 1497-1500 (1991).

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“cDNA” refers to a single- or a double-stranded DNA that is complementary to and derived from mRNA.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g. BLASTN for nucleotide sequences) can be used. The BLASTN program (for nucleotide sequences) uses as defaults a wordlength (W) of 11, an expectation (E) of 10, a cutoff of 100, M=5, N=−4, and a comparison of both strands. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide matches and an identical percent sequence identity when compared to the corresponding alignment generated by the preferred program.

(c) As used herein, “sequence identity” or “identity” in the context of two nucleic acid sequences makes reference to a specified percentage of nucleotides in the two sequences that are the same when aligned for maximum correspondence over a specified comparison window, as measured by sequence comparison algorithms or by visual inspection.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted herein, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tm can be approximated from the equation: Tm 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tm) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook and Russell 2001, for a description of SSC buffer). Often, a high stringency wash is preceded by a low stringency wash to remove background probe signal. For short nucleic acid sequences (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. Stringent conditions may also be achieved with the addition of destabilizing agents such as formamide. In general, a signal to noise ratio of 2× (or higher) than that observed for an unrelated probe in the particular hybridization assay indicates detection of a specific hybridization. Very stringent conditions are selected to be equal to the Tm for a particular nucleic acid molecule.

Very stringent conditions are selected to be equal to the Tm for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

“Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. Polymerase chain reaction (PCR) may be carried out in accordance with known techniques. See, e.g., U.S. Pat. Nos. 4,683,195; 4,683,202; 4,800,159; and 4,965,188. Where the nucleic acid to be amplified is RNA, amplification may be carried out by initial conversion to DNA by reverse transcriptase in accordance with known techniques.

The term “biomarker” is generally defined herein as a biological indicator, such as a particular molecular feature, that may affect or be related to NMD activity.

The phrase “corresponding NMD-insensitive isoform” indicates that the NMD-insensitive isoform is derived from the same gene as the NMD-sensitive isoform. In certain embodiments, isoforms from multiple genes may be examined. In such a situation, a NMD-sensitive and a NMD-insensitive isoform are analyzed for each gene.

The invention will now be illustrated by the following non-limiting Example.

### Example 1

### Abstract

Nonsense-mediated RNA decay (NMD) selectively degrades mutated and aberrantly processed transcripts harboring premature termination codons (PTC). Cellular NMD activity is typically assessed using exogenous PTC-containing reporters. Guided by the fact that NMD directly controls the levels of a suite of endogenous alternatively spliced transcripts, we developed a broadly applicable strategy to reliably and conveniently monitor changes in cellular NMD activity after overcoming several inherently problematic aspects of assaying endogenous NMD targets. Our new method was validated genetically in distinguishing NMD regulation from alternative splicing regulation. With this robust method, we tested a panel of widely used chemical inhibitors for their impacts on NMD and identified NMD-inhibiting stressors consistent with previous reports but also found that NMD inhibition was not a universal response to various cellular stresses. The high sensitivity and broad dynamic range of our method revealed a strong correlation between NMD inhibition, endoplasmic reticulum (ER) stress and polysome disassembly upon thapsigargin treatment in a temporal and dose-dependent manner. We found little evidence for the involvement of calcium signaling, which was previously reported as the mechanism underlying thapsigargin-induced NMD inhibition. Instead, consistent with studies reporting NMD inhibition via eIF2a phosphorylation, we found that of the three unfolded protein response (UPR) pathways activated by thapsigargin, only protein kinase RNA-like endoplasmic reticulum kinase (PERK) was required for NMD inhibition. Finally, we discovered that ER stress compounded TDP-43 depletion in the upregulation of TDP-43-repressed cryptic isoforms that have been implicated in the pathogenic mechanisms of amyotrophic lateral sclerosis and frontotemporal dementia.

### Results

To overcome the limitations associated with exogenous reporters, we aimed to develop a simpler assay based on endogenous NMD targets to track changes in cellular NMD activity. Assaying endogenous targets does not have the limitations inherent to exogenous reporters, including necessary secondary validations, construction and cumbersome delivery of the reporters, variability associated with reporter delivery and expression and hindered application in hard-to-transfect primary cells and animals. Furthermore, multiple endogenous targets can be assessed in parallel to improve the robustness of the assay, whereas the reporter approach usually deals with one exogenous target at a time. The concrete advantages of directly assaying endogenous targets are summarized in Table 1.

TABLE 1
Comparison between the Method Described Herein and Traditional NMD
Reporter Methods

| | Method Described | |
|---|---|---|
| | Herein | NMD reporters |
| Endogenous targets | Yes | No |
| Exogenous targets | No | Yes |
| Target delivery | — | plasmid transfection or virus |
| | | infection |
| Generalized applications | broad | transfectable cell lines in which the |
| | | reporter promoter is active |
| Application in primary cells | Easy | Difficult |
| Application in tissue organs | Easy | Difficult |
| Application in animals | Easy | Difficult |
| Standardization of the | MIQE provided | ? |
| method | | |
| Prior preparation | primer design and | reporter design and construction, |
| | validation | validation and delivery |
| Assay throughput | parallel assessment of | assess one NMD target per sample |
| | multiple NMD targets and | with Northern blot, RT-qPCR, |
| | non-NMD control targets | fluorescence or bioluminescence |
| | per sample with RT-qPCR | assay |
| Linear dynamic ranges | Yes | No |
| provided (to assess precision | | |
| and accuracy of expression | | |
| quantitation) | | |
| Simplicity | ++ | |
| Secondary validation with | optional | necessary |
| endogenous targets | | |
| Sources of variability in | — | the degree of reporter |
| addition to sample variation | | overexpression, the transfection |
| | | method, transfection efficiency, the |
| | | quantity and quality of reporter |
| | | DNA |
| Signal-to-noise ratio | +++ | + |
| Reproducibility | ++ | + |
| Assay time | short | long |
| Cost | $ | $$ |

Monitoring NMD activity through endogenous NMD targets is inherently challenging. Although many genes have altered expression levels in NMD-deficient cells, any observed changes could also result from transcriptional regulation and thus unreliably reflect NMD activity. In mammalian cells it is mostly unclear which gene transcripts are directly degraded by NMD and whether their NMD regulation is cell context independent. Therefore, these genes can be used as secondary confirmation for changes in NMD activity but would be a poor readout in scenarios lacking reliable primary validation. These genes are also not generalizable for unbiased screening because of the overwhelming variables affecting their transcription.

To more effectively distinguish NMD regulation from transcriptional control, we designed our assay based on isoform-centric quantitation instead of gene-centric quantitation. A transcript with a stop codon >50 nts upstream of an exon-exon junction is consistently selected by NMD for degradation (Maquat et al., 2010. Cell 142: 368-374; Lykke-Andersen et al., 2000. Cell 103: 1121-1131). Such an RNA structure may result from alternative RNA splicing shifting the reading frame and resulting in a PTC. We focused on alternative isoforms that include or skip a short cassette exon to minimize the sequence difference between the two isoforms. We deliberately excluded alternative 5′ UTR, alternative 3′ UTR and intron retention to avoid complications reflecting differential translation efficiency or miRNA targeting. Because transcriptional regulation should occur the same for both NMD and non-NMD isoforms of a given gene, the effects of transcriptional regulation can be better separated from those of NMD regulation.

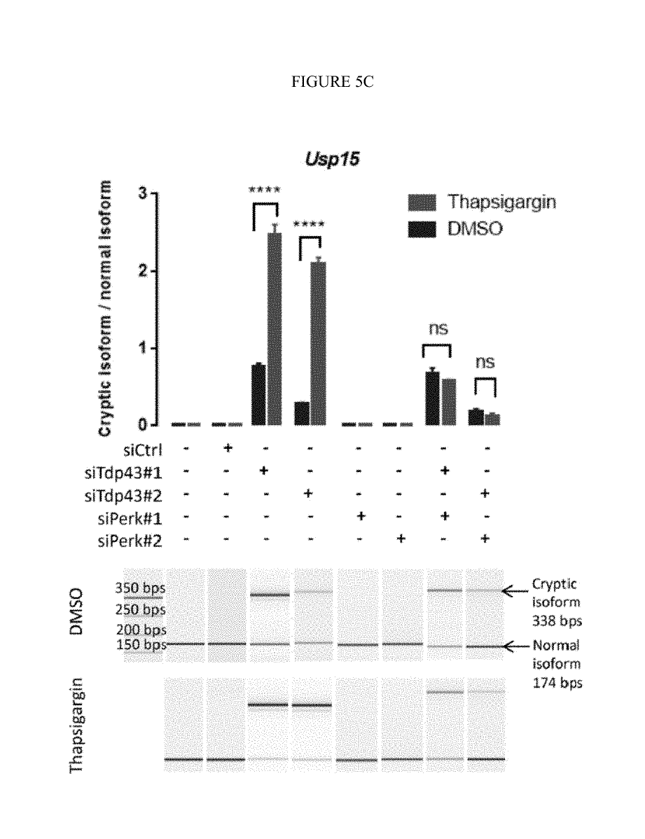

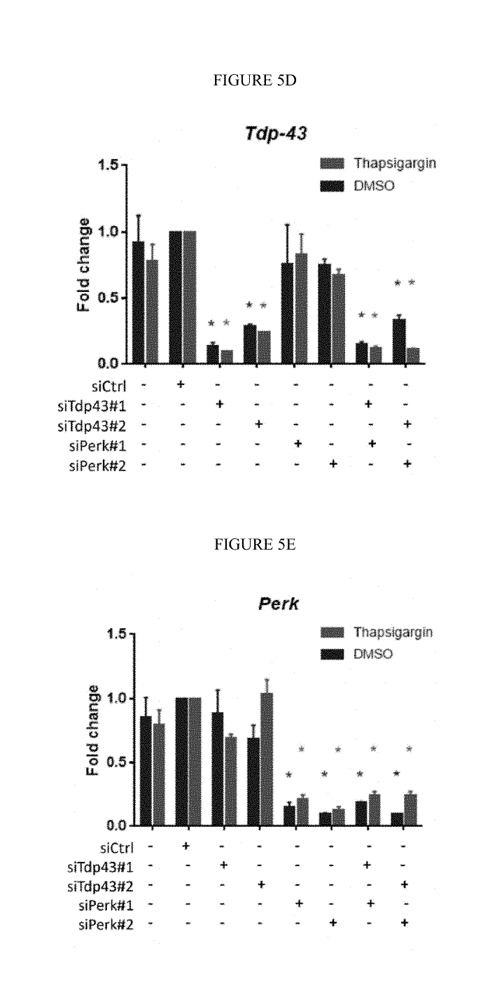

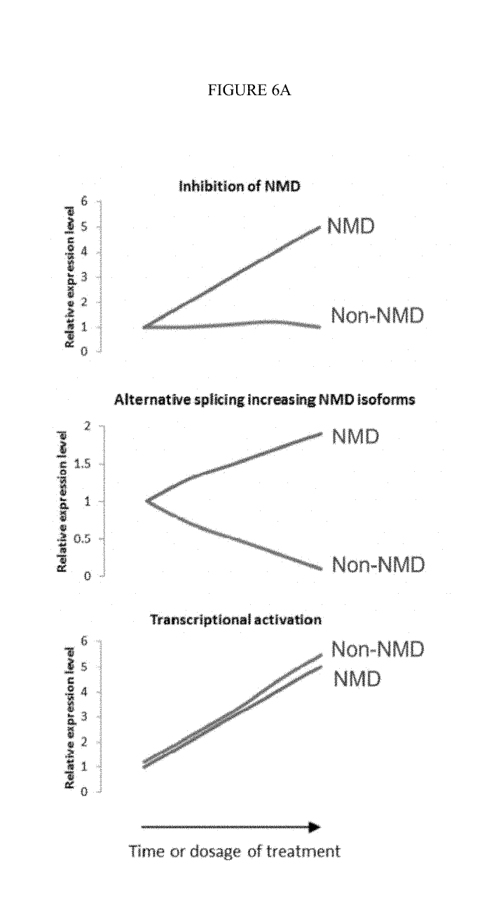

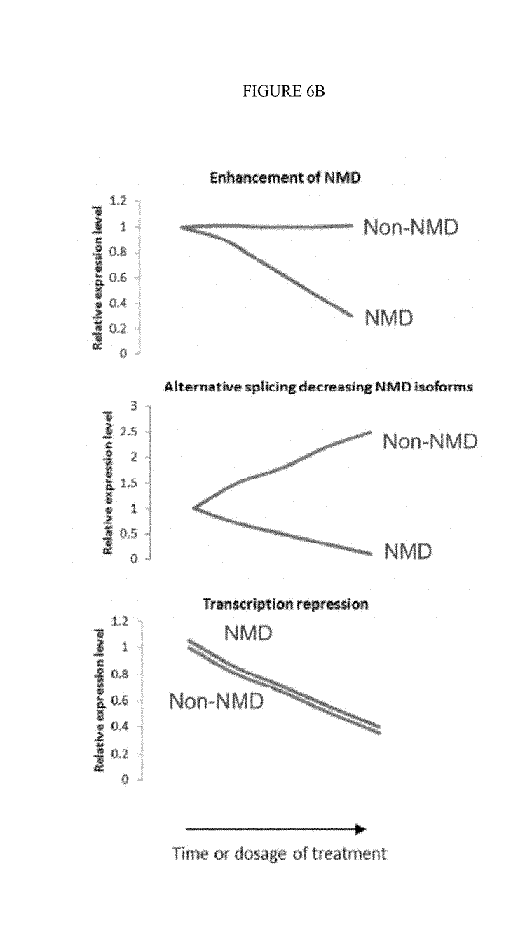

In our method, we measure the individual abundance of NMD-sensitive isoforms and that of their non-NMD counterparts via quantitative real-time PCR (qPCR). The NMD isoforms are first examined for differential expression between a treatment and a control condition. An increase in a NMD isoform may be due to NMD inhibition, transcriptional activation or a change in alternative splicing favoring the NMD isoform. These three scenarios can be distinguished by examining the expression of the non-NMD counterparts. Genuine NMD regulation, transcriptional activation and alternative splicing regulation would lead to no change, upregulation and down-regulation of the NMD-insensitive isoforms, respectively (FIG. 6A). Similarly, a decrease in a NMD isoform can be interpreted as a result of enhanced cellular NMD activity if the non-NMD isoform exhibits no change (FIG. 6B).

To enhance the robustness of our method, we included a suite of known endogenous NMD isoforms instead of relying on one single NMD target. To allow further improvement and standardization of the method by the community, we followed the guidelines of the international Real-time PCR Data Markup Language (RDML) consortium and provided detailed information of our quantitative real-time PCR experiments (FIG. 10 and Methods).

### Development of an Assay for Quantitative Monitoring of the Changes in Cellular NMD Activity

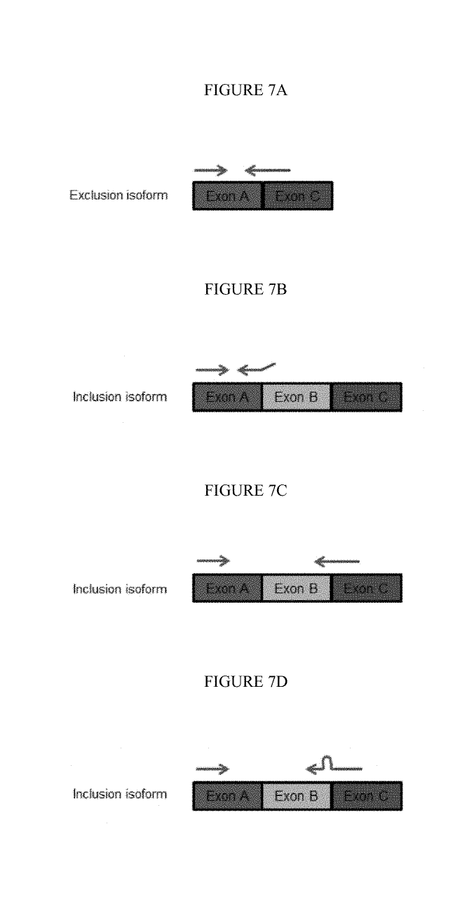

One technical difficulty of our method is designing RT-qPCR primers specific to the cassette exon-skipping isoform, whose entire sequence is contained in the inclusion isoform. To specifically detect an inclusion isoform, we designed a primer entirely annealing to the cassette exon (exon B in FIG. 1A). For the exclusion isoform, junction primers annealing to the exon-skipping junction appear to be the only choices. This can be either a reverse primer (FIG. 1A) with its 5′ portion matching the downstream constitutive exon (exon C) and its 3′ portion matching the upstream constitutive exon (exon A) or a forward primer (not shown) with its 5′ and 3′ portions matching the upstream and downstream exons, respectively. The challenge is herein illustrated with reverse primers but also applies to forward primers. Many exon-skipping junction primers are able to amplify the inclusion isoforms (FIGS. 7A-D). The longer the 3′ portion of the junction primer annealing to the upstream constitutive exon, the easier the primer amplifies the inclusion transcripts (FIG. 7B). Some exon-skipping junction primers with 3′ portions as short as six nucleotides, as we've observed, can still anneal to and detect the inclusion isoform at 55-60° C., albeit at a lower efficiency than its detection of the exclusion isoform. On the other hand, a longer 5′ portion and a shorter 3′ portion increase the possibility of the primer annealing to the exon B-exon C junction because of the sequence similarity around 5′ splice sites (FIGS. 7C-D). We therefore had to identify NMD-associated cassette exons containing a 3′ end unlike that of its upstream exon. We then screened multiple exon-skipping junction primers for one that amplified only the exclusion isoform and not the inclusion isoform. All primer pairs were tested and confirmed for their specificity, RT-qPCR efficiency and linear dynamic ranges (FIG. 10). Finally, two stably expressed housekeeping genes, Gapdh and Sdha, were used as internal controls for normalization by geometric averaging (Vandesompele et al., 2002. Genome Biol 3: RESEARCH0034).

The postsynaptic density protein 95 (Psd-95, Dlg4) gene encodes a scaffold protein, and its expression is regulated by polypyrimidine tract binding protein (PTBP) and NMD (Zheng et al., 2012. Nat Neurosci 15: 381-8, S1). Psd-95 is transcribed in many cells including embryonic stem cells (Zheng S. 2016. Int J Dev Neurosci. dx.doi.org/10.1016/j.ijdevneu.2016.03.003). PTBP1 inhibits exon 18, leading to a frameshift of Psd-95 transcripts, which are then targeted by NMD. The inclusion of exon 18 yields the non-NMD isoform. Because the two isoforms differ by only a small cassette exon and are identical for the remaining sequence, they should be subject to the same regulation other than NMD. Changes in NMD activity should alter the abundance of the NMD isoform but not the abundance of the non-NMD isoform. This is in contrast to the effects of altered transcriptional regulation, which elevates or lowers the levels of both isoforms in the same direction, as well as changes in alternative splicing, which alter the amount of the two isoforms in the opposite directions. Therefore, measuring the individual levels of the NMD and non-NMD isoforms from the same gene can be used to effectively infer NMD activity.

Our method is different from an alternative splicing assay that simultaneously amplifies both isoforms in one RT-PCR, with primers in the flanking constitutive exons, then resolves the two isoforms by electrophoresis and derives an expression ratio between the inclusion and exclusion isoforms. Such a semi-quantitative assay has been used to confirm AS-NMD targets but has not been used on its own to monitor NMD activity because it cannot definitively distinguish NMD regulation from alternative splicing regulation. For example, this assay could not discriminate between regulation induced by Upf1 RNAi and PTBP1 overexpression. Upf1 knockdown by siRNA #1 stabilized the exon 18-skipping Psd-95 NMD isoform (FIG. 1B). The ratio of the exclusion isoform to the inclusion isoform increased from around 0.6 in control cells to 2.0 in siUpf1(#1)-treated cells. Meanwhile, PTBP1 promotes exon 18 skipping. As a result, the ratio between the exclusion and inclusion isoforms also increased from 0.6 in control cells to 2.0 in PTBP1-overexpressing cells. Given a gel image or isoform ratios from the alternative splicing analysis (FIG. 1B), it is not possible to differentiate NMD regulation from alternative splicing regulation. In contrast, our method measuring isoform-specific expression via RT-qPCR effectively discriminated NMD regulation from alternative splicing regulation. When differential expression of the NMD isoform is detected, the expression of the non-NMD isoform is subsequently scrutinized to determine whether and how the two isoforms are differentially regulated. In cells deprived of Upf1, the non-NMD isoform exhibited no change, whereas in cells overexpressing PTBP1, the non-NMD isoform significantly decreased (FIG. 1C). RT-qPCR is typically more sensitive with a broader dynamic range than conventional alternative splicing assays. Note that in the alternative splicing assay, Upf1 knockdown with siRNA #2 for 48 hours induced a marginal insignificant change in the isoform ratio (FIG. 1B). However, with the same samples, our new method was sensitive enough to confirm significant upregulation of the NMD isoform (FIG. 1C).

One advantage of assessing endogenous NMD targets over exogenous PTC reporters is the applicability in hard-to-transfect cells and tissue organs (Table 1). Furthermore, the use of alternative splicing assays to measure NMD regulation in these hard-to-transfect samples becomes particularly challenging without corroboration from reporter assays. In contrast, our method can be used on its own to effectively monitor cellular NMD activity in these cases. For example, the ratio between the Psd-95 exclusion and inclusion isoforms increased from 0.21 in control wild-type cortices to 1.7 in Upf2^{lowP/lowP}; Emx1-cre (UPF2-cKO) cortices (FIG. 1E, left panel). This ratio decreased to 0.11 and 0.02 in Ptbp2^{+/-} and Ptbp2^{-/-} cortices, respectively (FIG. 1E, right panel). Without prior knowledge of the sample identification, it would be impossible simply based on these numbers to attribute the observed ratio changes to either NMD regulation or alternative splicing regulation. Without relying on the ratio analysis, our method effectively distinguished NMD regulation from alternative splicing regulation. In the conditional Upf2^{loxP/loxP}; Emx1-cre cortices, the exon 18-inclusion isoform exhibited no change, whereas in the Ptbp2 cortices, the levels of the inclusion isoform increased twofold relative to wild-type (FIG. 1F-G).

To further improve the robustness and specificity of our assay, we added other known AS-NMD targets including heterogeneous nuclear ribonucleoprotein L (Hnrnpl), serine/arginine-rich splicing factor 11 (Srsf11, Sfrs11), transformer 2 beta (Tra2b) and polypyrimidine tract binding protein 2 (Ptbp2, nPTB, brPTB). The NMD transcript isoforms of these genes are as follows: Hnrnpl including exon 6, Srsf11 including exon 2, Tra2b including exon 2 and Ptbp2 excluding exon 10 (Spellman et al., 2007. Mol Cell 27: 420-434; Saltzman et al., 2008. Mol Cell Biol 28: 4320-4330; Stoilov et al., 2004. Hum Mol Genet 13: 509-524; Boutz et al., 2007. Genes Dev 21: 1636-1652). These are all small cassette exons that moderately distinguish the two isoforms at the sequence level. Monitoring different genes that are targeted by NMD upon either exon inclusion or exon skipping for consistent NMD regulatory patterns was intended to exclude false positives that globally affect splicing. These genes were included in the final assay also because RT-qPCR primers specific to their exclusion isoforms were successfully identified. These five genes encode very different proteins and have been studied in various cell lines and tissues. They are widely transcribed and are not known to be transcriptionally coupled, making them suitable for broadly monitoring NMD activity.

### Thapsigargin is a Potent Inhibitor of Cellular NMD

Because our method can be used on its own can to infer NMD regulation with a low false positive rate, one of its applications is unbiased screening for changes in cellular NMD activity. We screened a small panel of widely used pharmacological inhibitors for their effects on NMD. The expression of both the NMD and non-NMD isoforms of Ptbp2, Srsf11, Tra2b, Hnrnpl and Psd-95 were simultaneously measured before and after drug treatment. When the levels of all of the NMD isoforms were altered by a chemical in the same direction, the expression of the NMD and non-NMD isoforms under the treatment and control conditions was then subjected to ANOVA analysis to determine differential regulation of the two isoforms. We were interested in drugs that affected the NMD isoforms across the board and more strongly than the non-NMD counterparts. With this criterion, we identified thapsigargin as having potent activity in blocking cellular NMD.

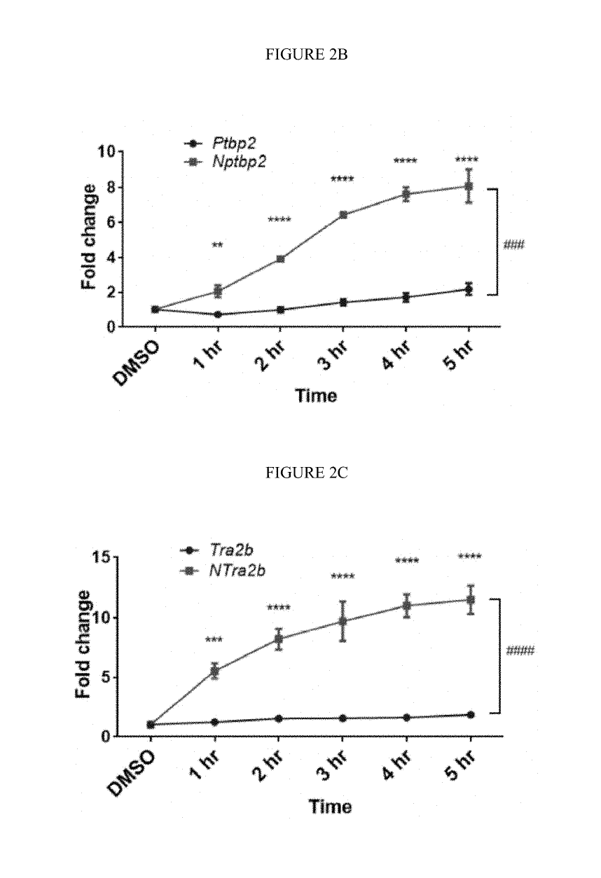

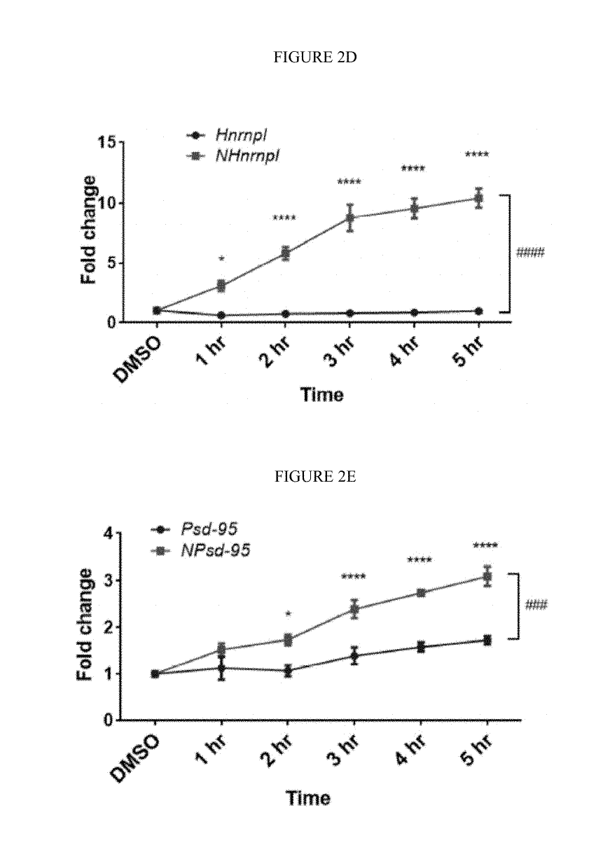

The abundance of the NMD isoform transcripts increased as early as 1 hour after thapsigargin treatment and continued to increase gradually (FIGS. 2A-E). At 5 hours, the NMD isoform levels were enhanced by 3 to 10 fold. In contrast, the non-NMD isoform levels barely changed for Srsf11, Tra2b and Hnrnpl. The non-NMD isoforms of Ptbp2 and Psd-95 increased slightly to around 1.8 fold. Although transcription and alternative splicing regulation might have modestly contributed to the changes in Ptbp2 and Psd-95, the dramatic increase in the levels of the NMD isoforms for all five genes could not be attributed solely to transcriptional stimulation or splicing changes. Rather, the changes were consistent with attenuation of the decay pathway specific to these isoforms, i.e., NMD.

### Our Method Revealed a Strong Correlation Between ER Stress, Polysome Disassembly and NMD Inhibition in a Thapsigargin Dose-Dependent Manner

Thapsigargin is a non-competitive inhibitor of the sarco/endoplasmic reticulum Ca²⁺ ATPase (SERCA) (Wictome et al., 1992. Biochem J 283 (Pt 2): 525-529; Lytton et al., 1991. J Biol Chem 266: 17067-17071). Thapsigargin treatment increases intracellular Ca²⁺ concentration, which stimulates various Ca²⁺ dependent signaling pathways. In fact, Nickless et al. recently reported that intracellular calcium inhibited NMD after administration of thapsigargin (Nickless et al. 2014. Nat Med 20: 961-966). Thapsigargin also induces ER stress. NMD inhibition in response to some stresses was shown to be mediated by phosphorylation of eukaryotic initiation factor 2alpha (eIF2α) (Gardner L B. 2008. Mol Cell Biol 28: 3729-3741; Wang et al., 2011. Mol Cell Biol 31: 3670-3680), although the exact mechanism underlying NMD inhibition by eIF2a phosphorylation remains to be determined (Karam et al., 2015. EMBO Rep 16: 599-609). The diverging mechanisms reported by these studies were both based on the traditional NMD reporter approach and gene-centric validation. We therefore investigated which of the proposed mechanisms our independent method would support. We first tested whether core NMD factors were affected at 5 hours after thapsigargin treatment and found no changes in their expression (data not shown).

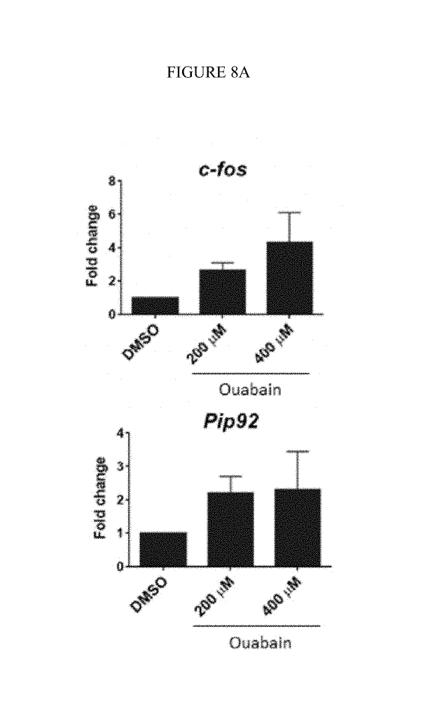

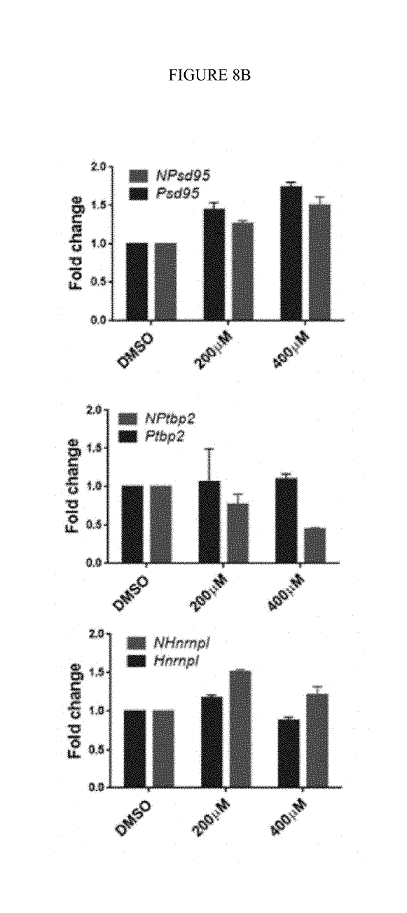

We then examined whether an increase in intracellular Ca²⁺ is sufficient to inhibit NMD. We applied a wide dose range of ouabain, a cardiac glycoside used by Nickless et al., and measured the abundance of both NMD and non-NMD isoforms at various time-points. However, only minimal changes in cellular NMD activity were observed (FIGS. 8A-B). Even at concentrations as high as 200 or 400 μM that clearly stimulated the immediate early genes c-fos and Pip92 (Chung et al., 2001. J Biol Chem 276: 2132-2138; Peng et al., 1996. J Biol Chem 271: 10372-10378; Nakagawa et al., 1992. J Biol Chem 267: 8785-8788), the NMD isoforms were not substantially enhanced (FIGS. 8A-B). To further test whether increased intracellular Ca²⁺ was sufficient to inhibit NMD, we treated the cells with ionomycin, a Ca²⁺ ionophore, as another method of raising the cytoplasmic Ca²⁺ concentration. Cells were responsive to ionomycin, with increased expression of the immediately early genes c-Jun and Pip92. However, ionomycin treatment in a range of 1 to 100 μM did not alter the levels of the NMD isoforms. Taken together, the results of ouabain and ionomycin treatment suggested that enhanced cytosolic Ca²⁺ signaling was not the major mechanism underlying the inhibitory action of thapsigargin in N2a cells.

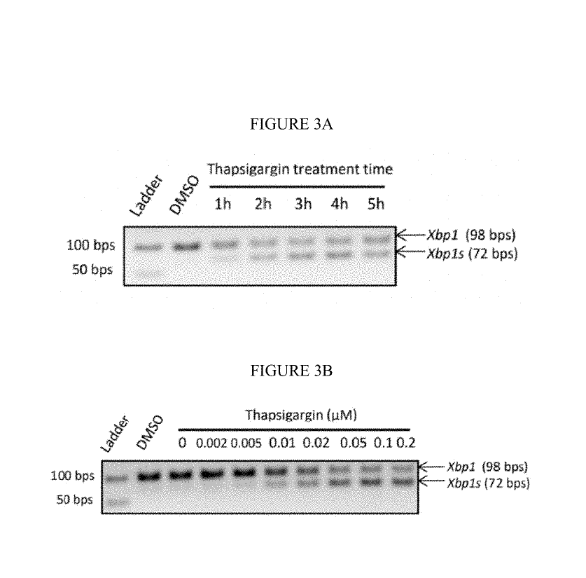

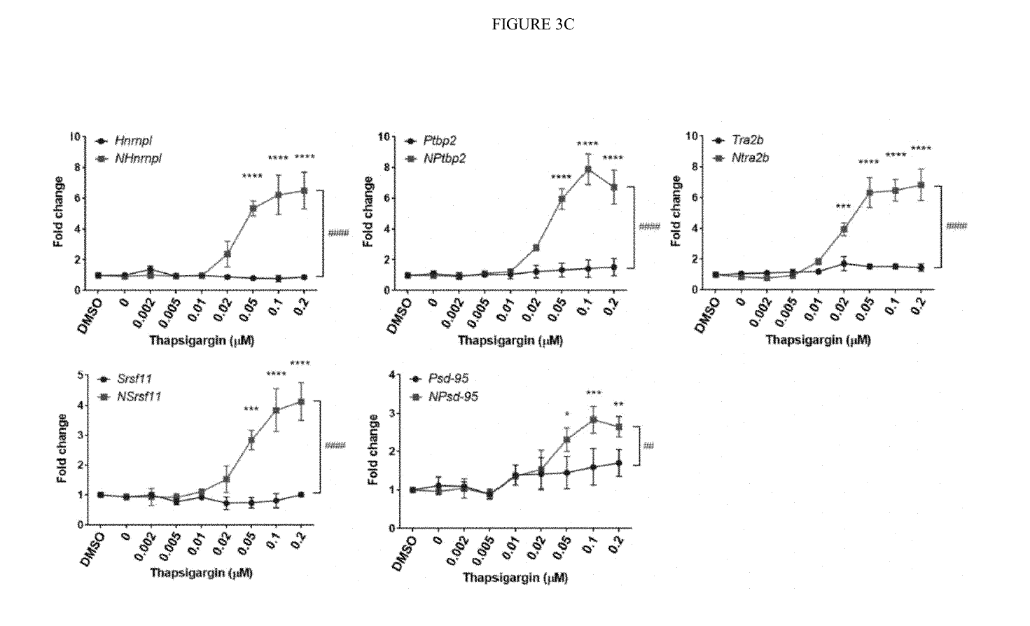

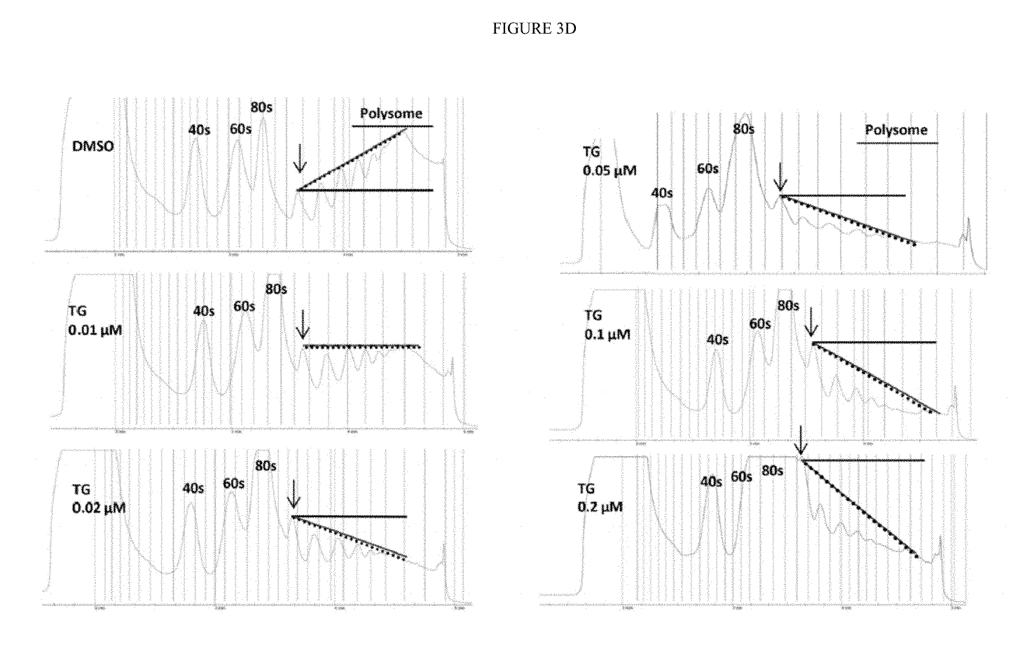

We investigated another consequence of thapsigargin treatment, ER stress, for its possible association with NMD inhibition. Because thapsigargin-induced NMD inhibition occurred as early as 1 hour post-treatment, we examined cellular stress levels by measuring the splicing activity of X-box binding protein 1 (Xbp1). Xbp1 splicing rapidly responds to ER stress. As a part of the unfolded protein response (UPR) triggered by ER stress, the serine/threonine protein kinase/endoribonuclease inositol-requiring enzyme 1 (IRE1α) oligomerizes and activates its ribonuclease activity through trans-autophosphorylation (Rubio et al., 2011. J Cell Biol 193: 171-184; Samali et al., 2010. Int J Cell Biol 2010: 830307; van Schadewijk et al., 2012. Cell Stress Chaperones 17: 275-279; Hetz C. 2012. Nat Rev Mol Cell Biol 13: 89-102). Activated IRE1α excises a 26-nt intron of Xbp1 mRNA, resulting in a shorter isoform (Xbp1s) that encodes a potent transcriptional activator for the expression of chaperones. RT-PCR with primers flanking the 26-nt intron detected Xbp1 splicing at 1 hour after thapsigargin treatment (FIG. 3A). The intron excision was continuously enhanced thereafter, mirroring the kinetics of NMD inhibition. Since IRE1α is targeted by NMD (Karam et al., 2015. EMBO Rep 16: 599-609; Oren et al. 2014. EMBO Mol Med 6: 685-701), the Xbp1 intron excision could have been due to thapsigargin-induced NMD inhibition. However, we observed almost no lag time between NMD inhibition and Xbp1 intron excision after thapsigargin treatment, suggesting that the two events happened almost simultaneously in the early phase. Nevertheless, feedback between these two pathways may have occurred in the later phase (see below).

To further evaluate the association between ER stress and NMD inhibition, we titrated the doses of thapsigargin and reexamined NMD activity and ER stress levels. Cells were treated with thapsigargin at concentrations of 0.002, 0.005, 0.01, 0.02, 0.05, 0.1 and 0.2 μM and harvested at 5 hours post-treatment for analysis. Xbp1 intron excision occurred at 0.01 μM and intensified with increasing concentration (FIG. 3B). NMD inhibition, collectively and consistently demonstrated by the expression of Srsf11, Ptbp2, Tra2b, Hnrnpl and Psd-95, was observed at a concentration as low as 0.02 μM and escalated in a dose-dependent manner (FIG. 3C). Xbp1s splicing and NMD repression exhibited the same kinetics, and both plateaued at 0.1 and above (FIG. 3B-C). Concentrations above 0.2 μM did not further increase NMD inhibition (data not shown). This analysis supported a strong positive correlation between ER stress and NMD inhibition and indicated a higher sensitivity of Xbp1 intron excision than of NMD inhibition to thapsigargin treatment.

In addition to altering gene expression via the IRE1-Xbp1s signaling pathway to retain homeostasis, ER stress also reduces global protein synthesis (Harding et al., 1999. Nature 397: 271-274; Ron D. 2002. J Clin Invest 110: 1383-1388; Wek et al., 2006. Biochem Soc Trans 34: 7-11). The dose of thapsigargin inhibiting NMD was as low as 20 nM, whereas according to the literature, thapsigargin has been widely used at 1 μM to induce ER stress. To assess whether the NMD-inhibiting thapsigargin doses also repressed translation, we performed ribosome fractionation using sucrose density gradient centrifugation and examined polysome integrity as an indicator of translational activity. Control cells treated with DMSO showed a typical polysome profile consisting of peaks of individual ribosome subunits (40S and 60S), monosomes (80S) and polysomes (2, 3, 4, 5 and >6 ribosomes). Under the culture conditions used, the heights of the polysome peaks steadily increased with the number of ribosomes (FIG. 3D). Thapsigargin effectively reduced the heights of the polysome peaks. The heavier polysomes (>4 ribosomes) were affected the most and further collapsed with increasing doses of thapsigargin. Polysome disintegration was also accompanied by increasing optical density values of 80S. To quantify the dose effect of thapsigargin, we drew a line from the peak of the disome to the peak of the polysome consisting of roughly 8 ribosomes based on relative elution time. We measured the angle of this line relative to a horizontal line through the disome peak and used the angle as an indicator of polysome integrity. This angle was 30 for DMSO-treated cells and decreased to 0, −18, −20, −30 and −40 in response to increasing thapsigargin concentrations of 0.01, 0.02, 0.05, 0.1 and 0.2 μM, respectively. Interestingly, 0.01 μM thapsigargin induced noticeable disintegration of heavy polysomes but little NMD repression. These data showed that polysome disassembly was positively associated with and also appeared to lead NMD inhibition.

### Thapsigargin Inhibits NMD by Activating the PERK Pathway

Since the degree of NMD inhibition was strongly correlated with the extent of ER stress measured by Xbp1 splicing and polysome disassembly, we next checked which signaling pathway directly led to NMD inhibition. ER stress stimulates three branches of the unfolded protein response (UPR): the IRE1, activating transcription factor 6a (ATF6α) and protein kinase RNA-like endoplasmic reticulum kinase (PERK) pathways (Hetz C. 2012. Nat Rev Mol Cell Biol 13: 89-102; Yoshida et al., 2001. Cell 107: 881-891; Calfon et al., 2002. Nature 415: 92-96; Harding et al., 1999. Nature 397: 271-274; Han et al. 2013. Nat Cell Biol 15: 481-490; Yoshida et al., 1998. J Biol Chem 273: 33741-33749). These three stress sensors bind to chaperone protein BIP and are quiescent under non-stress conditions. Under ER stress, misfolded proteins sequester BIP from continually interacting with these three proteins. Upon release from BIP binding, PERK is activated via autophosphorylation, similar to IRE1a. ATF6a is activated by intramembrane proteolysis and translocates into the nucleus to induce the transcription of chaperones, such as Bip. To test which pathway mediates NMD activity, we knocked down the stress sensors by RNAi for 48 hours before thapsigargin treatment and examined whether the thapsigargin-inhibited NMD activity could be restored.

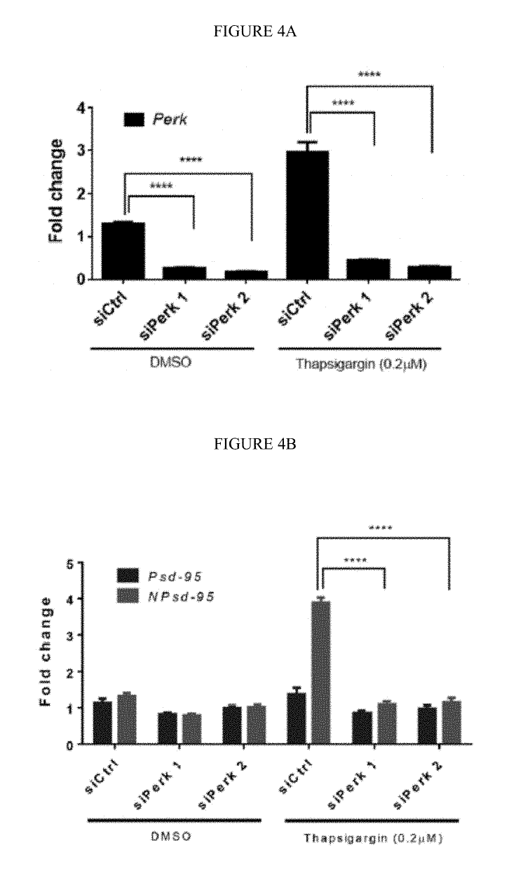

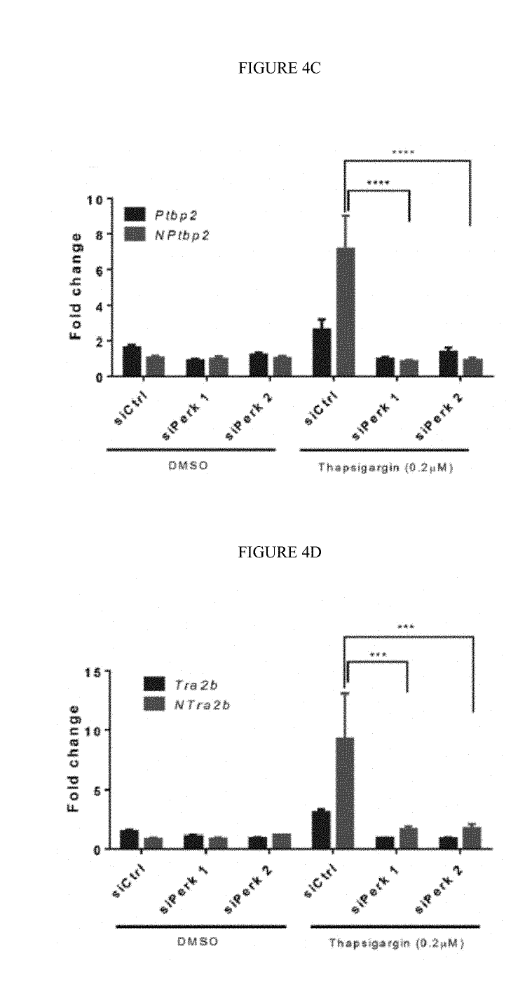

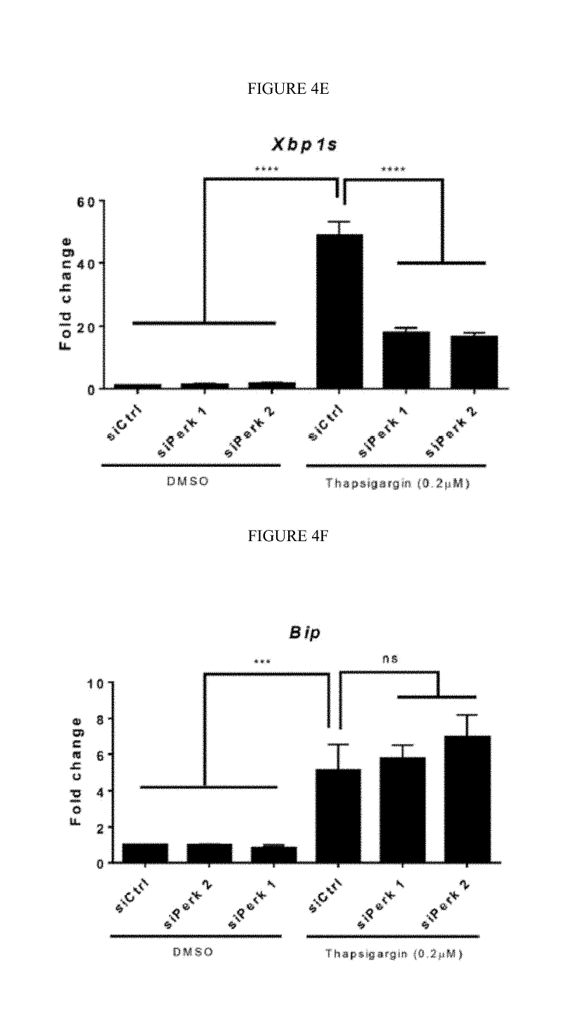

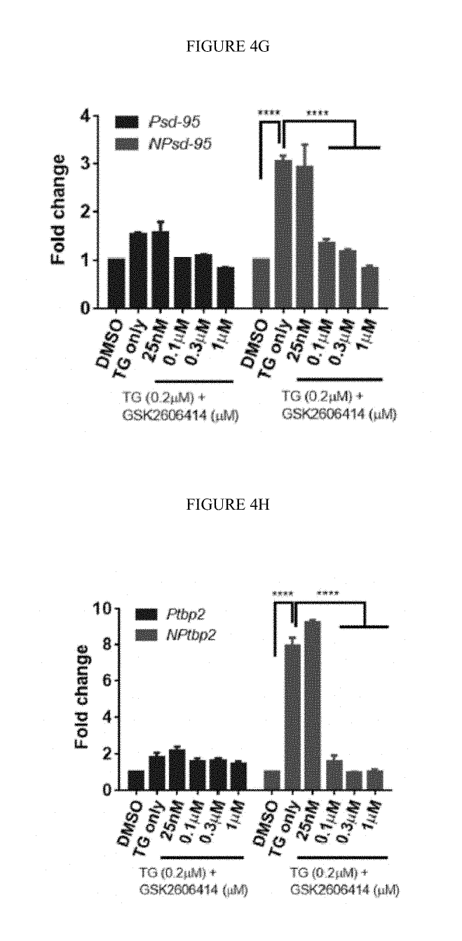

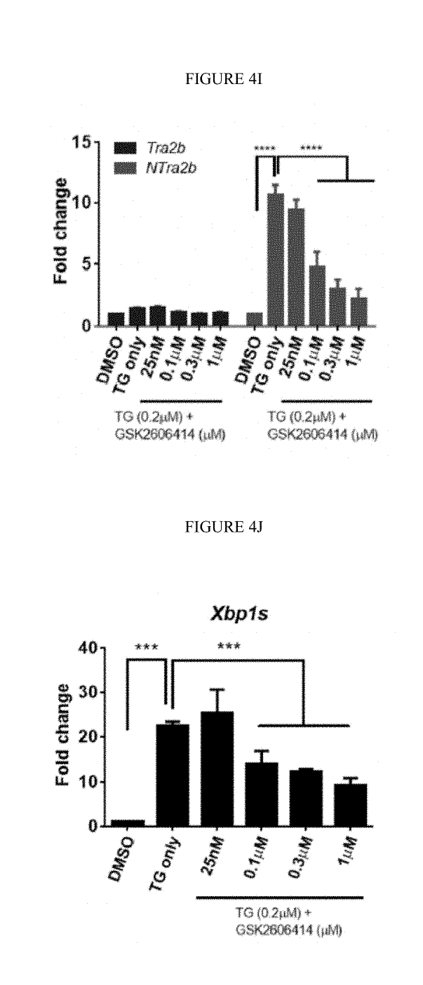

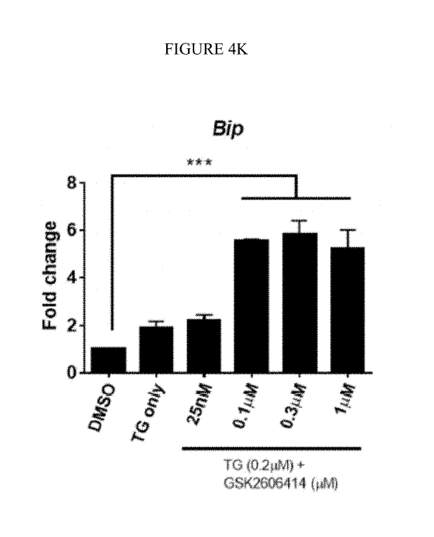

We found that depletion of only PERK completely prevented thapsigargin from repressing NMD. Two independent siRNAs reduced the endogenous Perk transcripts to around 20% (FIG. 4A). PERK knockdown did not significantly affect the steady-state levels of either the NMD or non-NMD isoforms of Psd-95, Ptbp2 and Tra2b (FIG. 4B-D). Thapsigargin application in control siRNA-pretreated cells induced only the NMD isoforms of these genes along with Xbp1s as previously demonstrated (FIG. 4B-E) and also interestingly increased Perk transcript levels (FIG. 4A). In siPerk-pretreated cells, none of the NMD isoforms were stimulated by thapsigargin (FIG. 4B-D).

We also found that activation of IRE1α and ATF6α by thapsigargin was not sufficient to inhibit NMD. Perk knockdown attenuated thapsigargin-induced Xpb1s (FIG. 4E) probably due to the restoration of NMD activity and because NMD inhibits IRE1α to splice Xbp1 (Oren et al. 2014. EMBO Mol Med 6: 685-701; Karam et al., 2015. EMBO Rep 16: 599-609). However, Perk knockdown did not completely repress Xpb1s. The Xbp1s level in the thapsigargin-treated siPerk cells was still 6 to 8 fold higher than in DMSO-treated cells, indicative of potent IRE1a activity. We also examined Bip transcript levels as a reporter of ATF6α activity (FIG. 4F). Control siRNA, Perk siRNAs and DMSO treatment did not change Bip expression. Thapsigargin treatment significantly boosted Bip transcript levels (FIG. 4F), which were maintained even with siPerk application. Therefore, Perk knockdown did not interfere with ATF6α activity but nevertheless prevented inhibition of NMD. In summary, in PERK-deficient cells, thapsigargin continued to activate IREα and ATF6α but no longer inhibited NMD.

Because RNAi-mediated depletion could not resolve whether it was the physical scaffold or enzymatic activity of PERK that was essential for NMD inhibition, we tested a small molecule inhibitor that inhibits only the enzymatic activity of PERK. GSK2606414 is a potent selective PERK inhibitor (Axten et al. 2012. J Med Chem 55: 7193-7207), and we applied it along with thapsigargin treatment. Application of GSK2606414, similar to siPerk alone, did not affect NMD activity in DMSO-treated cells. In thapsigargin-treated cells, however, GSK2606414 effectively reversed the upregulation of the NMD isoforms in a dose-dependent manner (FIG. 4G-I). GSK2606414 phenocopied Perk siRNA treatment in attenuating thapsigargin-induced Xbp1s but to a level still well above that in DMSO-treated cells (FIG. 4J). Like RNAi depletion of Perk, GSK2606414 also did not down-regulate Bip transcripts (FIG. 4K). These data further show that of the three UPR branches, only PERK activity was required for thapsigargin to inhibit NMD.

### NMD Inhibition is not Ubiquitous Under Various Cellular Stresses

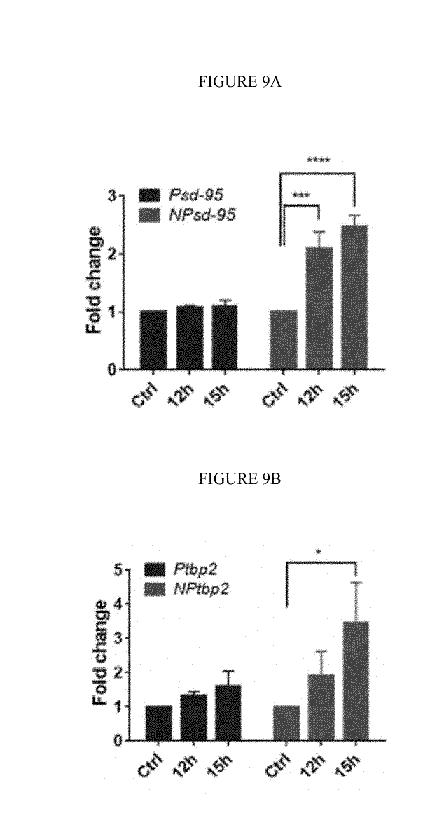

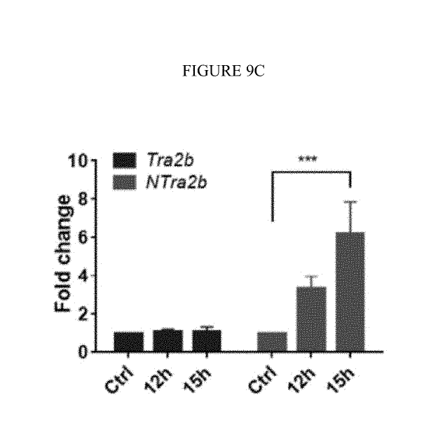

We then investigated whether NMD inhibition was ubiquitous under various cellular stresses. We did not observe changes in NMD isoform levels in cells subjected to high temperatures (up to 45° C. for 2 hours) or serum deprivation (as low as 0% fetal bovine serum for up to 24 hours). However, NMD inhibition was observed after culturing N2a cells in L-glutamine-free media (FIGS. 9A-C). The NMD isoform levels of Psd-95, Ptbp2 and Tra2b were not apparently altered within the first 6 hours of switching to L-glutamine-free media, possibly due to residual cellular glutamine sustaining cell metabolism. These NMD isoforms were clearly upregulated at 12 hours and further enhanced at 15 hours. These data show that induced NMD inhibition was not limited to stress caused by thapsigargin but also not ubiquitous under all cellular stresses.

### ER Stress Enhances TDP-43-Controlled Cryptic Isoforms Through PERK

The modulation of NMD activity may modify disease outcomes. RNA binding protein TDP-43 is commonly found in the cytoplasmic inclusion bodies of amyotrophic lateral sclerosis (ALS) and frontotemporal dementia (FTD), and its genetic mutations are linked to familial ALS and FTD (Neumann et al. 2006. Science 314: 130-133; Arai et al. 2006. Biochem Biophys Res Commun 351: 602-611; Guo et al. 2011. Nat Struct Mol Biol 18: 822-830; Ling et al., 2013. Neuron 79: 416-438; Polymenidou et al., 2012. Brain Res 1462: 3-15). Increased cryptic splicing in TDP-43-deficient cells has been proposed as one of the pathogenic mechanisms (Ling et al., 2015. Science 349: 650-655). These cryptic isoforms are presumably subject to NMD regulation. We therefore reasoned that ER stresses might aggravate TDP-43 deficiency in the upregulation of these cryptic isoforms.

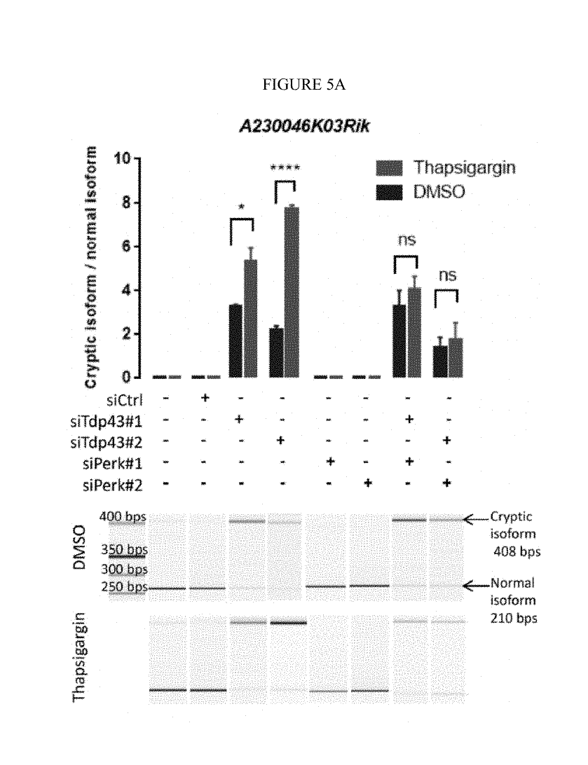

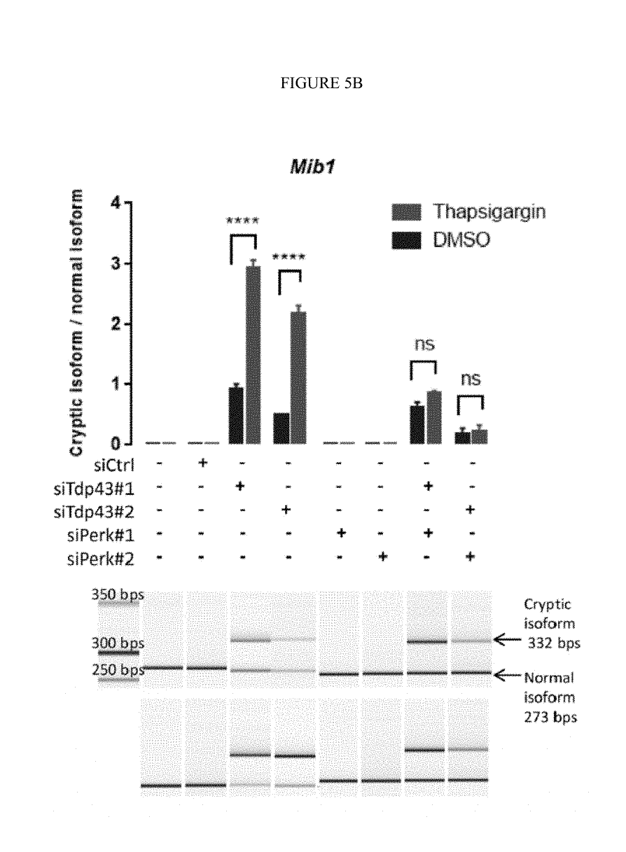

To investigate the possible compounding effect of ER stress on TDP-43-mediated cryptic isoforms, we examined the expression of TDP-43-repressed cryptic exons with and without thapsigargin treatment. We designed specific PCR primers flanking the previously reported cryptic exons of A230046K03Rik, Mib1 and Usp15 and performed capillary electrophoresis to measure the ratios of the cryptic isoforms to the normal isoforms. The splicing of cryptic exons was not detected in cells treated with control siRNA or DMSO but was drastically boosted by RNAi-mediated depletion of Tdp-43 (FIGS. 5A-E). Subsequent thapsigargin treatment further increased the level of the cryptic isoforms in TDP-43-deficient cells but had no effect in the mock-transfected or control siRNA-transfected cells.

To test whether the thapsigargin activity was due to PERK-mediated NMD inhibition, we knocked down Perk prior to the drug treatment. As shown by two independent Perk siRNAs, loss of PERK did not cause cryptic splicing on its own nor interfere with the activity of TDP-43 in DMSO-treated cells (FIGS. 5A-E). However, PERK knockdown completely eliminated thapsigargin's additive effects to TDP-43 depletion, resulting in similar isoform ratios between the DMSO and thapsigargin treatments. These results confirmed that ER stress exacerbated the upregulation of cryptically spliced NMD isoforms through PERK activation in TDP-43-deficient cells.

### Discussion

In this study, we devised a new method to precisely monitor changes in cellular NMD activity through the quantitative independent measurement of a panel of endogenous NMD isoforms and their corresponding non-NMD isoforms. Because the method can be used on its own to assess changes in cellular NMD activity, it is particularly suitable for unbiased screening. The assay was designed to distinguish NMD regulation from transcriptional regulation and alternative splicing control, which also affect the steady-state levels of NMD substrates. Transcriptional regulation should have the same effect on the two alternative isoforms including or skipping the cassette exon, while alternative splicing affects both isoforms in the opposite directions. In contrast to both transcriptional regulation and alternative splicing, NMD regulation should affect only the NMD isoform. Therefore, these distinct regulatory processes can be distinguished. Furthermore, we included five different NMD isoforms to enhance the robustness of this new method. The data for these five isoforms were always consistent in each of our experiments, indicating that fewer may suffice. Using this new method as an independent assay to traditional PTC reporters and gene-centric quantitation, we demonstrated that thapsigargin-induced NMD inhibition occurred via PERK activation rather than Ca′ signaling. We further showed that NMD inhibition was not universal under all cellular stresses.

Our new method is both sensitive and quantitative thanks to the high sensitivity and broad dynamic range of RT-qPCR. This was important to determining the strength and dynamics of NMD activity, analyzing the kinetics of drug response and dissecting the underlying molecular mechanisms. While the repressive effect of thapsigargin on NMD started to plateau at a concentration of 0.2 μM and above, NMD inhibition was already detected after treatment of as low as 0.02 μM. At this low concentration, the isoform upregulation was only about 20% of the maximal upregulation but was nevertheless consistently detected by our method (FIGS. 3A-D). Similarly, in the time course experiment, 20-30% of full inhibition (typically achieved at 5 hours post-treatment) was readily detectable at 1 hour post-treatment (FIGS. 2A-E). Our method also indicated that different NMD targets appeared to have different sensitivity to NMD inactivation for unknown reasons.

Among the three UPR branches, only the PERK signaling pathway but not the activation of IRE1α or ATF6α was required for thapsigargin-induced NMD inhibition. PERK activation phosphorylates eIF2α at serine 51, leading to attenuation of protein synthesis (Wek et al., 2006. Biochem Soc Trans 34: 7-11; Harding et al., 1999. Nature 397: 271-274). Our results demonstrated the necessity of PERK activation as well as a strong correlation between polysome disassembly and NMD inhibition. The findings were therefore consistent with previous reports that eIF2α phosphorylation inhibited NMD in the tumor microenvironment and under hypoxia (Gardner L B. 2008. Mol Cell Biol 28: 3729-3741; Wang et al., 2011. Mol Cell Biol 31: 3670-3680; Gardner L B. 2010. Mol Cancer Res 8: 295-308). We further showed that the other two branches of UPR signaling were neither necessary nor sufficient for NMD inhibition, supporting the hypothesis that NMD inhibition is specific to PERK-eIF2α-translation repression signaling. The present finding that disinhibition of NMD attenuated IRE1α activity is consistent with previous reports that NMD directly targets the IRE1α gene (Karam et al., 2015. EMBO Rep 16: 599-609; Oren et al. 2014. EMBO Mol Med 6: 685-701).

Our studies did not detect a role of intracellular Ca²⁺ signaling in NMD inhibition. Downstream of thapsigargin-induced Ca²⁺ release from the ER, PERK inhibition via a specific drug inhibitor or siRNA depletion did not reverse the increase in cytosolic Ca²⁺ but did completely restore NMD activity. Furthermore, other chemicals increasing intracellular Ca²⁺ failed to attenuate NMD. Therefore, increased intracellular Ca²⁺ was not enough to inhibit NMD in our system, contradicting a recent study reporting calcium's sufficient role (Nickless et al. 2014. Nat Med 20: 961-966). One possible explanation is the type of cells used for the analysis. We used mouse N2a cells, whereas Nickless et al. used human osteosarcoma cells (U2OS). Additionally, Nickless et al. used exogenous fluorescent mini-gene reporters to measure NMD activity, which would benefit from confirmation with endogenous NMD substrates.

With the new method, we also found that glutamine deprivation but not heat shock or serum withdrawal inhibited NMD. This result was consistent with a previous study reporting NMD attenuation upon deprivation of all amino acids (Mendell et al., 2004. Nat Genet 36: 1073-1078). Although the mechanism of NMD inhibition by amino acid starvation remains unclear, it is probably due to translation interference. Amino acid starvation activates general control nonderepressible 2 (GCN2), which can phosphorylate eIF2α (Dever et al., 1992. Cell 68: 585-596) to slow down translation (Pain V M. 1994. Biochimie 76: 718-728).

As a proof-of-principle analysis, our study demonstrated the utility of a new quantitative method for accurately monitoring changes in cellular NMD activities. The high sensitivity and broad dynamic range of our method allow for the detection of continual changes in NMD activity during development or in response to environmental changes. This presents a new avenue for exploring endogenous NMD modulation and subsequent applications in the study of genetic diseases. In the present analysis, we only tested a small panel of widely used small molecule inhibitors to demonstrate the potential of our assay for unbiased screening. With the aid of catalogued libraries, high-throughput robotic liquid handling systems and next-generation sequencing, our new method can be adapted to screen larger libraries.

### Materials and Methods

### Cell Cultures and Treatments

Neuro-2a (N2a) cells were maintained in N2a complete media consisting of L-glutamine-free Dulbecco's modified Eagle's medium (DMEM), 10% FBS and 1× GlutaMAX at 37° C. Thapsigargin (VWR, cat. no. 89161-410), ionomycin (Fisher Scientific, cat. no. AG-CN2-0416-M001) and ouabain octahydrate (Fisher Scientific, cat. no. 80055-364) were dissolved in DMSO and stored at −80° C. For the application of these chemicals, 750,000 cells were plated on 35 mm BioLite TC plates and incubated with 2 ml of N2a complete media overnight before treatment with the drugs at the indicated concentrations. The cells were collected at 5 hours post-treatment unless otherwise specified. The PERK inhibitor GSK2606414 (Thermo Fisher, cat. no. 501016108) was dissolved in DMSO and stored at −20° C. N2a cells were incubated with GSK2606414 for 1 hour before thapsigargin treatment. For L-glutamine deprivation, 750,000 N2a cells were plated overnight and switched to L-glutamine-free DMEM with 10% FBS. SiRNA knockdown experiments were conducted using Lipofectamine RNAiMax and Silencer® Select siRNAs (siTdp-43, cat. no. s106688 and s106686; siPerk, cat. no. s201280 and s65405; siUpf1, cat. no. s72879 and s72878) according to the manufacturer's instructions. Silencer® Negative Control siRNA (AM4615) was used as the siRNA control. Lipofectamine 2000 (Life tech) was used to transfect the Flag-Ptbp1 plasmid and control GFP plasmid into N2a cells. Cells were incubated for 48 hours for optimal knockdown efficiency or overexpression before downstream treatments.

### Animals

Conditional Upf2^{-/-} (that is, Upf2^{loxP/loxP}; Emx1-cre) mice were generated by first breeding Upf2^{loxP/loxP} mice to Emx1-cre mice and subsequently breeding Upf2^{loxP/+}; Emx1-cre mice to Upf2^{loxP/loxP} mice (Zheng et al., 2012. Nat Neurosci 15: 381-8, 51). Ptbp2^{-/-} mice were generated by breeding Ptbp2^{+/-} mice to Ptbp2^{+/-} mice (Li et al., 2014. Elife 3: e01201). All animal procedures were approved by the Institutional Animal Care and Use Committee at UCR.

RNA Extraction, cDNA Synthesis and RT-qPCR

Trizol (Life Technologies, cat. no. 15596-018) was directly added to the cells or mouse brain tissues to extract total RNA following the Trizol Reagent standard protocol. Isolated RNA was treated with 4 units of Turbo DNase (Ambion) at 37° C. for 35 minutes to degrade all remaining genomic DNA. After the DNase treatment, RNA was purified using phenol-chloroform (pH 4.5, VWR cat. no. 97064-744). RNA concentrations were measured using a Nanodrop 2000c (Thermo Fisher). One microgram of freshly isolated DNA-free RNA was converted to cDNA using 1 μl random hexamers (30 μM) and 200 units of Promega M-MLV reverse transcriptase (cat. no. M1705) following the Promega protocol in a 20 μl reaction. For all qPCR primers, quality control was performed for their specificity, sensitivity, melting curves and standard curves (FIG. 10). RT-qPCR experiments were conducted using a QuantStudio 6 Real-Time PCR instrument with 2× Power SYBR Green PCR master mix (Life Tech) following the Life Tech protocol. Each 10 μl reaction contained 0.3 μl cDNA, 5 μl 2× Power SYBR Green PCR master mix, 0.3 nM forward primer and 0.3 nM reverse primer. The QuantStudio 6 RT-qPCR run program was as follows: 50° C. for 2 minutes; 95° C. for 15 seconds and 60° C. for 1 minute, with the 95° C. and 60° C. steps repeated for 40 cycles; and a melting curve test from 60° C. to 95° C. at a 0.05° C./s measuring rate. QuantStudio Real-Time PCR software was used for the analysis. All RT-qPCR reactions were conducted with three technical replicates along with a no template control (NTC, not amplified). Outliers were excluded when the coefficient of variation of Ct for the three technical replicates was larger than 0.3. Relative expression (fold changes) was calculated using the ΔΔCt method. For the splicing assays of the NMD exons, PCR was performed using New England Biolab Taq DNA polymerase (cat. no. M0267E). All statistical analysis was performed using GraphPad Prism 6.

### Quantitative Analysis of Cryptic Exon Splicing

We optimized the PCR cycle numbers for Psd-95 (28 cycles), A230046K03Rik, Mib1 and Usp15 and eventually used 29 cycles for relative quantification of the transcript isoforms. Capillary gel electrophoresis of PCR amplicons was conducted using the QIAxcel Advanced System. The molar abundance (nM) of each isoform was quantified using QIAxcel Screengel software v1.4. Splicing of the cryptic exon was determined using the following formula. All statistical analysis was performed using GraphPad Prism 6.

$$Psd \text{-} 95 splicing ratio = \frac{NMD exon skipping isoform ( nM )}{non \text{-} NMD exon inclusion isoform ( nM )}$$

$$Cryptic splicing ratio = \frac{isoform including the cryptic exon ( nM )}{isoform including the cryptic exon ( nM )}$$

### Polysome Fractionation

For polysome fractionation, 1.5×10⁷ N2a cells in 20 ml N2a complete media were plated on 150 mm petri dishes overnight and treated with varying concentrations of thapsigargin the next day. Cycloheximide (Fisher Scientific, cat. no. 50490338) was added at a concentration of 100 μg/ml, and the cells were incubated for 10 min at 37° C. before lysate collection. The cells were washed twice with 10 ml cold 1×PBS containing 100 μg/ml cycloheximide then collected in 4 ml of the same cold PBS solution. The cells were lysed with 0.5 ml lysis buffer (20 mM Tris pH 7.5, 100 mM KCl, 5 mM MgCl₂, 2 mM DTT, 100 μg/ml cycloheximide, 1% Triton X-100, 50 u/ml RNaseout and 1×EDTA-free protease inhibitor cocktail). Roughly 400 μl (6,000 optical units) of lysate was loaded onto premade sucrose gradients (60% to 15%) and balanced (within 0.5 mg) before ultracentrifugation at 4° C. and 237,000 g (50,000 rpm for a SW55 Ti rotor) for 1.5 hours. Products were carefully removed from the ultracentrifuge and fractionated with the apparatus consisting of the gradient fractionator (Brandel SYN-202), the ISCO absorbance detector (ISCO # UA-6, Lincoln, Nebr.) and the fraction collector (R1 Fraction Collector) at 2.0 sensitivity and 150 cm/h chart speed to record absorbance data and collect fractionations.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of detecting and/or quantitating nonsense mediated RNA decay (NMD) activity in a cell, comprising measuring the RNA expression level of at least one NMD-sensitive isoform and at least one corresponding NMD-insensitive isoform in the cell, wherein the isoforms are derived from an endogenously expressed, alternatively spliced gene, and wherein an altered level of RNA expression of the NMD-sensitive isoform and an unaltered level of RNA expression of the corresponding NMD-insensitive isoform, as compared to a control cell, is indicative of NMD activity.

2. The method of claim 1, wherein the level of RNA expression of the NMD-sensitive isoform in the cell is increased and the level of RNA expression of the NMD-insensitive isoform in the cell is unaltered as compared to a control cell.

3. The method of claim 1, wherein the level of RNA expression of the NMD-sensitive isoform in the cell is decreased and the level of RNA expression of the NMD-insensitive isoform in the cell is unaltered as compared to a control cell.

4. The method of claim 1, wherein RNA expression levels of NMD-sensitive and insensitive isoforms, which are derived from two or more endogenously expressed alternatively spliced genes, are detected/measured; and wherein a NMD-sensitive and a corresponding NMD-insensitive isoform are assayed for each gene.

5. The method of claim 1, wherein the endogenously expressed, alternatively spliced gene is Ptbp2, Hnrnpl, Srsf11, Tra2b and/or Psd-95, or any combination thereof.

6. The method of claim 1, wherein the cell is a mammalian cell.

7. The method of claim 1, wherein the RNA expression levels are detected/measured using real-time polymerase chain reaction (qPCR), real-time quantitative polymerase chain reaction (RT-qPCR), digital PCR, or high-throughput sequencing.

8. The method of claim 7, wherein one or more primers having at least 90% sequence identity to a primer as described in FIG. 10 is used in a RT-qPCR assay.

9. The method of claim 1, wherein the method is capable of distinguishing NMD activity from alternative splicing regulation and transcriptional regulation.

10. A method for screening a compound for nonsense mediated RNA decay (NMD) modulating activity, comprising
1) measuring the RNA expression level of at least one NMD-sensitive isoform and at least one corresponding NMD-insensitive isoform in a first population of cells, wherein the isoforms are derived from an endogenously expressed, alternatively spliced gene;
2) contacting a second population of cells with the compound; and
3) subsequently measuring the RNA expression level of the at least one NMD-sensitive isoform and the at least one corresponding NMD-insensitive isoform in the second population of cells;
wherein the compound has NMD modulating activity if i) the RNA expression level of the at least one NMD-sensitive isoform in the second population of cells is altered as compared to the first population of cells; and ii) the RNA expression level of the at least one corresponding NMD-insensitive isoform in the second population of cells is unaltered as compared to the first population of cells.

11. The method of claim 10, wherein the RNA expression level of the at least one NMD-sensitive isoform and the at least one corresponding NMD-insensitive isoform in the second population of cells is measured 1 or more hours after contact with the compound.

12. The method of claim 10, wherein the first and second populations of cells comprise the same cell type.

13. The method of claim 10, wherein the compound increases NMD activity.

14. The method of claim 10, wherein the compound decreases NMD activity.

15. The method of claim 10, wherein RNA expression levels of NMD-sensitive and insensitive isoforms, which are derived from two or more endogenously expressed alternatively spliced genes, are detected/measured; and wherein a NMD-sensitive and a corresponding NMD-insensitive isoform are assayed for each gene.

16. The method of claim 10, wherein the endogenously expressed, alternatively spliced gene is Ptbp2, Hnrnpl, Srsf11, Tra2b and/or Psd-95, or any combination thereof.

17. The method of claim 10, wherein the cell is a mammalian cell.

18. The method of claim 10, wherein the RNA expression levels are detected/measured using real-time polymerase chain reaction (qPCR), real-time quantitative polymerase chain reaction (RT-qPCR), digital PCR, or high-throughput sequencing.

19. The method of claim 18, wherein one or more primers having at least 90% sequence identity to a primer as described in FIG. 10 is used in a RT-qPCR assay.

20. The method of claim 1, wherein the isoforms are derived from an endogenously expressed, alternatively spliced gene selected from Ptbp2, Hnrnpl, Srsf11, Tra2b and/or Psd-95; and wherein the RNA expression levels are measured using real-time quantitative polymerase chain reaction (RT-qPCR).

21. A method of inhibiting nonsense mediated RNA decay (NMD) in a cell, comprising contacting the cell with an effective amount of thapsigargin, or a salt thereof.
